(19) United States
(12) Patent Application Publication
(10) Pub. No.: US 2011/0177043 A1
(43) Pub. Date: Jul. 21, 2011

(54) METHOD OF IMPROVING DIFFERENTIATION OF CHONDROGENIC PROGENITOR CELLS

(75) Inventors: Tongming Liu, Singapore (SG); Eng Hin Lee, Singapore (SG); Bing Lim, Singapore (SG)

(73) Assignee: Agency for science,technology and research(A*star), Singapore (SG)

(21) Appl. No.: 13/120,205

(22) PCT Filed: Sep. 22, 2009
(86) PCT No.: PCT/SG09/00351

§ 371 Date: Mar. 22, 2011

(30) Foreign Application Priority Data
Sep. 22, 2008 (UA) 61/192784

(51) Int. Cl. A61K 48/00; C12N 5/10; A61K 35/32; A61K 38/02; C12N 15/85

(52) U.S. Cl. 424/93.21; 435/377; 514/44 R; 514/1.1; 435/455

(57) ABSTRACT

We disclose a method of promoting cartilage, bone or ligament repair or inducing repair or regeneration of chondral tissue, the method comprising enhancing the expression or activity of ZNF145 or a fragment, homologue, variant or derivative thereof in an chondrogenic progenitor cell, for example a mesenchymal stem cell. We also provide for a chondrogenic progenitor cell, for example a mesenchymal stem cell (MSC) engineered to increase expression or activity of ZNF145 or a fragment, homologue, variant or derivative thereof.

### FIELD

The present invention relates to the fields of development, cell biology, molecular biology and genetics. More particularly, the invention relates to a polypeptide that promotes chondrogenesis of a chondrogenic progenitor cell, for example a mesenchymal stem cell.

### BACKGROUND

A major area in regenerative medicine is the application of stem cells in cartilage tissue engineering and reconstructive surgery. Stem cells can be distinguished from progenitor cells by their capacity for both self-renewal and multilineage differentiation, whereas progenitor cells are capable only of multilineage differentiation without self-renewal (3). It is this capacity for self-renewal that makes stem cells particularly useful for transplantation medicine. Stem cells for cartilage repair can be derived from two major sources: ES cells derived from the inner cell mass of blastocysts-stage embryos, and mesenchymal stem cells (MSCs).

The most obvious advantage of using ES cells for cartilage regeneration is that ES cells are immortal and could potentially provide an unlimited supply of differentiated chondrocytes and chondroprogenitor cells for transplantation in theory. However, the tendency of ES cells to spontaneously differentiate to multiple lineages and the low efficiency of directed differentiation of ES cells to the chondrogenic differentiation remains a major obstacle in their use for regenerative medicine. In addition, potential problems with immunogenecity and teratoma formation of ES cells within the transplant recipient also poses high risk in using these cells for tissue transplantation.

Mesenchymal stem cells (MSCs) are adult pluripotent stem cells present in the bone marrow, adipose tissue and umbilical cord blood, and other tissues, which contribute to the regeneration of mesenchymal tissues such as bone, cartilage, adipose, muscle, ligament, tendon and stroma (4-5). The methods and compositions described here may therefore be used for promoting cartilage repair or inducing repair or regeneration of such tissues.

One possible use of MSCs is in the orthopaedic context because of the clear demonstration of their ability to differentiate into bone and cartilage (6-9). Due to the differentiation potential of MSCs into bone and cartilage, and the relatively simple requirements for in vitro expansion and genetic manipulation, MSCs are one of the most promising stem cells types for cartilage repair. However, the self-renewal and proliferative capacity of MSCs is very much limited and seems to decrease with age (10-11). In addition, MSCs gradually lose their stem cell properties during ex vivo expansion. Major limitations in using MSCs for tissue engineering are in obtaining sufficient cells for transplantation. This would obviously limit their usefulness for the treatment of age-related degenerative diseases of cartilage such as osteoarthritis.

Putative MSCs from bone marrow is in fact a highly heterogenous population, with only a limited proportion of cells being capable of differentiating into the chondrogenic lineage. Instead they are made up of a heterogeneous population of both pluripotent stem cells and tripotent, bipotent and unipotent progenitors (12-13). In addition, variability across samples from different patients in cartilage differentiation also pose great inconvenience to their clinical application and elucidation of basic issues related to MSCs.

### SUMMARY

According to a 1^{st} aspect of the present invention, we provide a chondrogenic progenitor cell such as a mesenchymal stem cell (MSC) engineered to increase expression or activity of ZNF145 or a fragment, homologue, variant or derivative thereof.

The chondrogenic progenitor cell, for example a mesenchymal stem cell, may display enhanced expression of a chrondrogenic marker. The chondrogenic marker may comprise collagen type 2 (COL2A1). It may comprise aggrecan. It may comprise col10A1. It may comprise Sox 9.

The chondrogenic progenitor cell, for example a mesenchymal stem cell, may display enhanced secretion of cartilage proteoglycans. The enhanced secretion may be detected by alcian blue staining.

The chondrogenic progenitor cell, for example a mesenchymal stem cell, may display improved ability to repair a cartilage, bone or ligament defect. The enhanced ability may be detected by histological grading of any suitable marker. This may comprise one or more of cell morphology, matrix-staining, surface regularity, thickness of cartilage and integration of donor with host adjacent cartilage. The enhanced ability may be detected by histological grading as described by Wakitani et al (1994).

The chondrogenic progenitor cell, for example a mesenchymal stem cell, may display any combination of the above. The features set out above may be as compared to a chondrogenic progenitor cell, for example a mesenchymal stem cell, that has not been so engineered.

The chondrogenic progenitor cell, for example a mesenchymal stem cell, or an ancestor thereof may be transfected with an expression construct that increases the expression or activity of ZNF145 or a fragment, homologue, variant or derivative thereof. The expression construct may comprise a lentiviral expression construct.

The chondrogenic progenitor cell, for example a mesenchymal stem cell, may be induced to chondrocyte differentiation. It may be induced to chondrocyte differentiation by a pellet culture system such as described in Liu et al., 2007. The system may comprise pelleting chondrogenic progenitor cells, for example mesenchymal stem cells, and culturing in chondrogenic medium containing 10 ng/ml transforming growth factor (TGF)-β3, 10-7 M dexamethasone, 50 μg/ml ascorbate-2-phosphate, 40 μg/ml proline, 100 μg/ml pyruvate, and 50 mg/ml ITS+Premix (Becton Dickinson; 6.25 μg/ml insulin, 6.25 μg/m transferrin, 6.25 μg/ml selenious acid, 1.25 mg/ml BSA, and 5.35 mg/ml linoleic acid).

The chondrogenic progenitor cell, for example a mesenchymal stem cell, may be engineered to increase expression or activity of any one or more of the following: Nanog, Oct4, telomerase, SV40 large T antigen, HPV E6, HPV E7 and Bmi-1.

There is provided, according to a 2^{nd} aspect of the present invention, a cell line comprising or derived from a chondrogenic progenitor cell, for example a mesenchymal stem cell, as set out above. The chondrogenic progenitor cell, for example a mesenchymal stem cell line, may comprise an immortal or immortalised cell line.

We provide, according to a 3^{rd} aspect of the present invention, a nucleic acid comprising a ZNF145 sequence, or a fragment, homologue, variant or derivative thereof capable of encoding a polypeptide comprising chondrogenic activity for use in a method of treatment of a disease. The nucleic acid may comprise an expression vector. The disease may comprise repair or regeneration of chondral tissue, a disease, damage, disorder or injury associated with a cartilage, bone or ligament defect, a traumatic injury, an age-related degenerative disease or a degenerative joint disease.

As a 4^{th} aspect of the present invention, there is provided a polypeptide comprising a ZNF145 sequence, or a fragment, homologue, variant or derivative thereof comprising chondrogenic activity, for use in a method of treatment of a disease. The disease may comprise repair or regeneration of chondral tissue, a disease, damage, disorder or injury associated with a cartilage, bone or ligament defect, a traumatic injury, an age-related degenerative disease or a degenerative joint disease.

We provide, according to a 5^{th} aspect of the present invention, a pharmaceutical composition comprising a chondrogenic progenitor cell, for example a mesenchymal stem cell, as set out above, a cell line as set out above, a nucleic acid as set out above, or a polypeptide as set out above.

The present invention, in a 6^{th} aspect, a method comprising modulating the expression or activity of ZNF145 or a fragment, homologue, variant or derivative thereof in a chondrogenic progenitor cell, for example a mesenchymal stem cell. The method may comprise increasing expression or activity of ZNF145 or a fragment, homologue, variant or derivative thereof. Such an increase may promote chondrogenesis of a chondrogenic progenitor cell, for example a mesenchymal stem cell. The method may comprise down-regulating expression or activity of ZNF145 or a fragment, homologue, variant or derivative thereof Such a decrease may reduce chondrogenesis of an chondrogenic progenitor cell, for example a mesenchymal stem cell.

In a 7^{th} aspect of the present invention, provides a method of promoting cartilage, bone or ligament repair or inducing repair or regeneration of chondral tissue. The method may comprise enhancing the expression or activity of ZNF145 or a fragment, homologue, variant or derivative thereof in an chondrogenic progenitor cell, for example a mesenchymal stem cell.

According to an 8^{th} aspect of the present invention, there is provided use of an engineered chondrogenic progenitor cell, for example a mesenchymal stem cell, as set out above, a cell line as set out above, a nucleic acid as set out above, a polypeptide as set out above or a pharmaceutical composition as set out above, for the treatment of, or the preparation of a pharmaceutical composition for the treatment of, any one of the following: repair or regeneration of chondral tissue, a disease, damage, disorder or injury associated with a cartilage, bone or ligament defect, a traumatic injury, an age-related degenerative disease or a degenerative joint disease.

We provide, according to a 9^{th} aspect of the invention, we provide a method of treating a disease in an individual, the method comprising up-regulating the expression or activity of ZNF145 or a fragment, homologue, variant or derivative thereof in a chondrogenic progenitor cell, for example a mesenchymal stem cell, in or of the individual or administering a chondrogenic progenitor cell, for example a mesenchymal stem cell, that displays increased expression or activity of ZNF145 or a fragment, homologue, variant or derivative thereof to an individual in need of such treatment. The treatment may be for repair or regeneration of chondral tissue, a disease, damage, disorder or injury associated with a cartilage, bone or ligament defect, a traumatic injury, an age-related degenerative disease or a degenerative joint disease.

The chondrogenic progenitor cell, for example a mesenchymal stem cell, may comprise a feature as set out above.

There is provided, in accordance with a 10^{th} aspect of the present invention, use of ZNF145 or a fragment, homologue, variant or derivative thereof as a marker for chondrogenic differentiation of a chondrogenic progenitor cell, for example a mesenchymal stem cell.

As an 11^{th} aspect of the invention, we provide a method of modulating the expression or activity of Sox9, the method comprising modulating the expression or activity of ZNF145 or a fragment, homologue, variant or derivative thereof.

We provide, according to a 12^{th} aspect of the invention, there is provided a method of identifying an agent capable of enabling or promoting chondrogenesis of a chondrogenic progenitor cell, for example a mesenchymal stem cell, the method comprising contacting ZNF145 or a fragment, homologue, variant or derivative thereof with a candidate agent and determining whether the candidate agent binds to ZNF145 or a fragment, homologue, variant or derivative thereof, and optionally determining whether the expression or activity of ZNF145 or a fragment, homologue, variant or derivative thereof is thereby modulated.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 shows upregulation of ZNF145 during differentiation of MSCs into 3 lineages.

FIG. 1A. Expression of ZNF145 is quantified by real time PCR during differentiation into 3 lineages for 7 days, showing upregulation of ZNF145 during differentiation into 3 lineages.

FIG. 1B. Expression of ZNF145 is localized in nuclei by immunofluorescence during adipogenesis and osteogenesis for 7 days whereas ZNF145 is not expressed in control MSCs.

FIG. 1C. Expression of ZNF145 is localized in nuclei by immunofluorescence during chondrogenesis under pellet culture for 7 days whereas ZNF145 is not detected in control MSCs.

FIG. 1D. Expression pattern of ZNF145 during chondrogenesis of MSCs.

FIG. 2 shows the effect of small interfering RNA (siRNA)-mediated gene silencing of ZNF145 on differentiation of MSCs.

FIG. 2A. MSCs are infected with high efficiency by lentivirus with ZNF145 shRNA and GFP.

FIG. 2B. After 48 h of chondrogenesis of ZNF145-knockdown MSCs under pellet culture, efficiencies of reduction of ZNF145 and chondrogenic marker are measured by real time PCR compared with no insert control.

FIG. 2C. ZNF145 knockdown slowed down differentiation of MSCs into 3 lineages. MSCs are infected with ZNF145 shRNA and induced into adipogenesis for 14 days, chondrogenesis for 28 days and osteogenesis for 14 days, three lineages of markers are measured by real time PCR compared with no insert control.

FIG. 2D. ZNF145-knockdown and control MSCs are induced into adipogenesis for 14 d, chondrogenesis for 28 d and osteogenesis for 14 d. Effects of ZNF145 knockdown on differentiation of MSCs are assessed by oil red stain for intracellular lipid-filled droplets in adipogenesis, immunostaining for collagen type 2 and alcian blue stain for sulfated proteoglycan matrix in chondrogenesis and alirazin red stain for calcium deposits in osteogenesis, respectively. Compared to negative controls, ZNF145-knockdown MSCs showed a notable lower staining in all 3 differentiation pathways.

FIG. 3 shows effects of ZNF145 overexpression on the differentiation of MSCs

FIG. 3A. ZNF145 is overexpressed in nuclei of MSCs by lentiviral system whereas ZNF145 is not detected in undifferentiated MSCs.

FIG. 3B. ZNF145 overexpression improved differentiation of MSCs into cartilage and bone compared with no insert control. ZNF145 overexpressing MSCs are induced into osteogenesis for 14 days and chondrogenesis for 28 days, osteogenic and chondrogenic markers are measured by real time PCR compared with no insert control.

FIG. 3C. ZNF145 overexpression and control MSCs are induced into chondrogenesis for 28 d and osteogenesis for 14 d. Effects of ZNF145 overexpression on differentiation of MSCs are assessed by immunostaining for collagen type 2 and alcian blue stain for sulfated proteoglycan matrix in chondrogenesis and alirazin red stain for calcium deposits in osteogenesis. Compared to no insert controls, ZNF145 overexpressing MSCs showed enhanced staining in cartilage and bone differentiation pathways.

FIG. 3D and FIG. 3E. Enhanced alkaline phosphatase activity by AP assay upon ZNF145 overexpression in osteogenesis of MSC cell line. ZNF145 overexpressing and control MSC cell line is induced into osteogenesis for 14 days, alkaline phosphatase activity are quantified by AP assay compared with control.

FIG. 3D. AP stain shows enhanced alkaline phosphatase stain in osteogenesis by ZNF145 overexpression.

FIG. 3E. AP assay shows enhanced alkaline phosphatase activity in osteogenesis by ZNF145 overexpression.

FIG. 3F. ZNF145 overexpression enhanced chondrogenesis and osteogenesis of MSC cell line in vitro compared with no insert control. ZNF145 overexpressing MSC cell line is induced into chondrogenesis for 28 days and osteogenesis for 14 days, chondrogenic and osteogenic markers are measured by real time PCR compared with no insert control

FIG. 3G. ZNF145 overexpression improves chondrogenesis and osteogenesis of MSC cell line. ZNF145 overexpressing and control MSCs are induced into chondrogenesis for 28 d and osteogenesis for 14 d. Effects of ZNF145 overexpression on differentiation of MSC cell line are assessed by immunostaining for Col2A1 and alcian blue stain for sulfated proteoglycan matrix in cartilage and alirazin red S stain for calcium deposits in osteogenesis. Compared to no insert control, ZNF145 overexpressing MSC cell line shows enhanced staining for chondrogenic and osteogenic differentiation.

FIG. 3H. Enhanced alkaline phosphatase (AP) stain by ZNF145 overexpression during osteogenesis. ZNF145 overexpressing and control MSC cell line is induced into osteogenesis for 14 days, alkaline phosphatase activity was determined by AP stain.

FIG. 3I. Enhanced alkaline phosphatase activity by AP assay upon ZNF145 overexpression in osteogenesis of MSC cell line. ZNF145 overexpressing and control MSC cell line was induced into osteogenesis for 14 days, alkaline phosphatase activity were quantified by AP assay compared with control.

FIG. 4 shows global gene expression analyses by microarrays.

FIG. 4A. Pearson correlation analysis of 14312 probes is performed to cluster no insert control MSCs and ZNF145 overexpressing MSCs from two different individuals. Red indicates increased expression whereas green indicates decreased expression.

FIG. 4B. Genes upregulated in ZNF145 overexpressing MSCs. ZNF145 overexpressing MSCs from two patients showed similar expression profile in upregulated genes.

FIG. 4C. Verification of microarray data by RT-PCR. RT-PCR assays are consistent with the microarray data.

FIG. 5 shows Sox9 upregulation by ZNF145 overexpression in undifferentiated MSCs. MSCs are infected with lentivirus for overexpressing ZNF145 and Sox9, no insert is used as control. The results showed ZNF145 upregulated Sox9 whereas Sox9 did not regulated ZNF145, suggesting ZNF145 is an upstream regulator of Sox9. A, RT-PCR; B, Western blot analysis.

FIG. 6. ZNF145 improves osteochondral defect repair in a rat model.

FIG. 6A, FIG. 6B and FIG. 6C. The ZNF145-overexpressing and no insert control MSCs are induced into cartilage differentiation for 7 days under pellet culture and then pellets are transplanted into osteochondral defects of rat knees for 6 weeks. The results showed ZNF145 group showed better and earlier repair of the osteochondral defects than the no insert control group at 6 w.

FIG. 6A. 40× magnification. FIG. 6B. 100× magnification. FIG. 6C. Histological grading scale shows significant differences in repair of cartilage defects at 6 w between ZNF145 and no insert control group evaluated according to Wakatani et al. (1994) (*P<0.05).

FIG. 6D, FIG. 6E and FIG. 6F. The ZNF145-overexpressing and no insert control MSCs were induced into cartilage differentiation for 7 days under pellet culture and then pellets were transplanted into the osteochondral defects of rat knees for 12 weeks. The results showed ZNF145 group showed better repair and integration of osteochondral defect than no insert control group at 12 w.

FIG. 6D. 40× magnification. FIG. 6E. 100× magnification. FIG. 6F. Histological grading scale showed significant differences in repair of cartilage defects at 12 w between ZNF145 and no insert control group evaluated according to Wakatani et al. (1994) (*P<0.05).

### DETAILED DESCRIPTION

Our invention is based on the demonstration that ZNF145 has a role in the regulation of chondrogenic differentiation of chondrogenic progenitor cells such as mesenchymal stem cells.

We show that small interfering RNA-mediated gene silencing of ZNF145 results in a decrease in the expression of chondrogenic specific genes. Overexpression of ZNF145 increases expression of genes such as collagen type 2A1 (col2A1), aggrecan, SRY (sex determining region Y)-box 9 (Sox9) and collagen type 10A1 (col10A1). ZNF145 expression may therefore be used as a marker for chondrogenesis.

We demonstrate that targets of ZNF145 in undifferentiated MSCs include Sox9, cartilage linking protein 1 (HAPLN1) and alkaline phosphatase (ALPL), as determined by microarray. ZNF145 overexpression enhances expression of Sox9 whereas Sox9 overexpression does not affect the expression of ZNF145. This shows that ZNF145 regulates chondrogenesis as an upstream regulator of Sox9.

In the Examples, allogeneic transplant of ZNF145 over-expressing hMSCs into rat show that ZNF145 repairs cartilage defects much better than no insert control MSCs. These findings show that ZNF145 therapy may be used as a strategy for cartilage regeneration and repair. It may also be used for regeneration and repair of other tissues, such as bone and ligament.

ZNF145 in nucleic acid form or polypeptide form, agonists of ZNF145 capable of up-regulating its activity or expression and ZNF145 over-expressing cells such as mesenchymal stem cells may therefore be used as chondrogenesis-promoting agents, as described in this document.

### Chondrogenesis-Promoting Agent

The Examples indicate that ZNF145 and its agonists may be used to initiate, maintain or stabilise chondrogenesis in a chondrogenic progenitor cell, for example a mesenchymal stem cell.

We therefore provide for a number of chondrogenesis-promoting agents comprising any combination of ZNF145, an agonist thereof, and a ZNF145 over-expressing cell. The ZNF145 over-expressing cell may comprise a chondroprogenitor cell that is, or has been, engineered to over-express ZNF145. The ZNF145 over-expressing cell may comprise any suitable type of cell. It may comprise a mesenchymal stem cell. It may also comprise a cartilage cell, an umbilical cord stem cell, a bone marrow stromal cell, an adipose stromal cell or a chondrogenic progenitor cell derived from periosteum or synovium.

We provide for methods for inducing chondrogenesis comprising administering a therapeutically effective amount of a chondrogenesis-promoting agent as described herein, optionally together with a pharmaceutically acceptable carrier.

We describe a chondrogenesis-promoting agent comprising a ZNF145 nucleic acid. The chondrogenesis-promoting agent may comprise a ZNF145 polypeptide. We therefore provide for the use of ZNF145 nucleic acids and polypeptides in medicine, for example in treating a degenerative disease.

The methods described here may lead to cartilage formation. They may lead to cartilage formation that further mediates formation of new bone tissue in a vertebrate. The methods may be used for cartilage, ligament or bone generation, regeneration or repair. The methods may be used for any treatment in which any of these aims is desirable. Such treatment may be for disease, injury such as traumatic injury, damage, etc of cartilage, ligament or bone.

The chondrogenesis-promoting agent may comprise an agent capable of up-regulating the activity or expression of ZNF145. Agents capable of up-regulating the activity or expression of ZNF145 are referred to generally as ZNF145 agonists for the purposes of this document. In general, a ZNF145 agonist may comprise any chemical that binds to ZNF145 with a Kd of less than 1 micromolar. A ZNF145 agonist may comprise a chemical agent that enhances or elevates any one or more of the activities or functions of ZNF145, as described in detail below.

ZNF145 agonists include transcriptional, translational or post-translational activators of ZNF145. ZNF145 agonists also include molecules which enhance DNA binding or transcriptional activation activity (or both) of ZNF145 to its target sequence. ZNF145 agonists may be identified by testing or screening, as described in further detail below.

An example of an ZNF145 agonist is a ZNF145 expression vector. ZNF145 expression vectors may be used to up-regulate expression of ZNF145 in a cell, such as a mesenchymal stem cell. An example of an expression vector is one which comprises a regulatory sequence and a ZNF145 coding sequence. Any expression vector suitable for the host cell may be used. For example, a lentiviral expression vector capable of up-regulating expression of ZNF145 may be transfected into a cell such as a chondrogenic progenitor cell, for example a mesenchymal stem cell.

The chondrogenesis-promoting agent may be used to promote chondrogenesis of any suitable cell or cell type. The chondrogenesis-promoting agent may be used to promote chondrogenesis of a mesenchymal stem cell chondrocyte, a cartilage chondrocyte, an umbilical cord stem cell chondrocyte, a bone marrow stromal cell chondrocyte, an adipose stromal cell chondrocyte, a chondrogenic progenitor cell chondrocyte or a combination thereof.

The chondrocyte may be selected from the group consisting of hyaline cartilage chondrocytes, fibro-cartilage chondrocytes, elastic cartilage chondrocytes, juvenile articular chondrocytes, adult articular chondrocytes and a combination thereof. The chondrogenic precursors may be selected from the group consisting of synovial capsule chondrogenic progenitor cells, periosteum chondrogenic progenitor cells, embryonic stem cell chondrogenic progenitor cells and a combination thereof.

The chondrogenesis-promoting agent may be used to promote chondrogenesis of a chonrogenic progenitor cell. The chondrogenesis-promoting agent may be used to promote chondrogenesis of a mesenchymal stem cell.

The chondrogenesis-promoting agent may comprise a ZNF145 over-expressing cell. The cell may comprise a mesenchymal stem cell. The ZNF145 over-expressing cell may be used as a source of ZNF145 for treatment. It may be used to generate cartilage, which may be used for repair. The repair may be for cartilage, bone or ligament. Such a cell, for example, a mesenchymal stem cell in which expression of ZNF145 is up-regulated may itself be used as a medicament. A ZNF145 over-expressing cell may be produced by transfecting, transforming or otherwise causing entry of a ZNF145 expression vector into a cell such as a mesenchymal stem cell, culturing the mesenchymal stem cell and allowing ZNF145 to be expressed therefrom. A cell line may be derived from such a cell. The cell line may be transformed, or otherwise immortalised. Methods of immortalisation include expression of telomerase or one or more viral genes, as described in detail below and in the Examples.

An ZNF145 over-expressing cell or cell line which has been immortalised by engineering to express telomerase and/or a viral gene may be used for treatment or for the production of a pharmaceutical composition as described in this document.

The ZNF145 over-expressing mesenchymal stem cell may be administered to a patient in need of treatment. The mesenchymal stem cell which is transfected, etc with the expression vector may come from any source. For example, it may be taken from the same patient to which it is later administered (allogenic transplantation).

The chondrogenesis-promoting agents described in this document may be used generally in promoting chondrogenesis. They may be used to promote chondrogenesis in a cell, tissue, organ or individual. They may be used generally for the generation, repair or regeneration of chondral tissue. They may be used generally for cartilage generation, re-generation or repair. They may be used for bone generation, regeneration or repair. They may be used for ligament generation, regeneration or repair.

They may be used to treat a disease in which chondrogenesis is affected, deficient reduced, inhibited or otherwise impaired. In general, the methods may be used to treat any disorder associated with loss or damage to the structure or function of bone, cartilage or ligament.

The methods may be used in the treatment of a degenerative disease, such as a disease in which cartilage, bone or ligament is damaged or deficient, etc. They may be used to prevent or slow down the onset of such diseases.

Degenerative diseases are described in further detail below, and may include a disease associated with a cartilage defect, a bone defect, a ligament defect, an age-related degenerative disease or a degenerative joint disease.

The chondrogenesis-promoting agents may also be used in treating an injury in which cartilage, bone or ligament is damaged. Such an injury may include a traumatic injury or a sports injury. It may include traumatic damage.

The chondrogenesis-promoting agents described above may be administered in any combination to a patient in need of treatment. They may be provided in the form of pharmaceutical compositions, which may comprise any combination of one or more of the chondrogenesis-promoting agents.

We describe a method comprising the steps of: (a) isolating a cell such as a mesenchymal stem cell; (b) causing the cell to have the capability to over-express ZNF145, such as by transfecting a ZNF145 expression vector into the cell; (c) growing the cell in vitro; and (d) using the expanded cells to produce hyaline-like cartilage tissue or a population of cells that is useful for transplantation.

We describe a method of treating an individual comprising administering to the individual cells as described above, such as mesenchymal stem cells, collected from at least one donor. “Donor” as used herein means an adult, child, infant, or, preferably, a placenta. The method may comprise administering to an individual cells that are collected from a plurality of donors and pooled. The cells may be chondrogenic progenitor stem cells taken from a plurality of donors. When collected form multiple donors, the dosage units, where a “dosage unit” is a collection from a single donor, may be pooled prior to administration, may be administered sequentially, or may be administered alternatively.

We further describe a kit for generating cells for cartilage, bone or ligament repair, the kit comprising one or more of a ZNF145 nucleic acid, a ZNF145 polypeptide, a ZNF145 agonist and a ZNF over-expressing cell, together with instructions for use.

The kit may comprise a pharmaceutical pack. It may comprise one or more containers filled with one or more of the ingredients of the pharmaceutical compositions described here. Optionally associated with such container(s) can be: an apparatus for cell culture, one or more containers filled with a cell culture medium or one or more components of a cell culture medium, an apparatus for use in delivery of the compositions described here, e.g., an apparatus for the intravenous injection of the compositions of the invention, and/or a notice in the form prescribed by a governmental agency regulating the manufacture, use or sale of pharmaceuticals or biological products, which notice reflects approval by the agency of manufacture, use or sale for human administration. The kit may comprise one or more containers filled with a chondrogenic progenitor stem cell such as mesenchymal stem cells and one or more different containers filled with ZNF145 or an agonist thereof, as disclosed elsewhere in this document.

### Uses of ZNF145 Chondrogenesis-Promoting Agents

We provide compositions comprising such chondrogenesis-promoting agents, which may comprise any one or more of ZNF145, a ZNF145 agonist and a ZNF145 over-expressing cell.

We describe therapeutic compositions and uses of such compositions to treat disorders involving abnormal tissue formation, including cartilage formation, bone formation and ligament formation. The therapeutic compositions may be used for the treatment of disorders involving abnormal cartilage, ligament or bone formation and associated abnormal skeletal development resulting from disease or due to trauma.

The therapeutic compositions may comprise an effective amount of an chondrogenesis-promoting agent. They may be provided in the form of a pharmaceutical composition that further includes a pharmaceutically acceptable carrier. The chondrogenesis-promoting agents comprise a stimulating effect on cartilage, bone or ligament formation and may result in associated bone development in a vertebrate.

The chondrogenesis-promoting agent may be used in a method for stimulating cartilage, bone or ligament formation in a vertebrate. Such a method may comprise administering to the vertebrate an effective cartilage, bone or ligament formation stimulating amount of an chondrogenesis-promoting agent. It may be used in a method for treating damaged cartilage, bone or ligament and associated bone in a subject. Such a method may comprise administering to the subject an effective amount of an chondrogenesis-promoting agent. The chondrogenesis-promoting agent may stimulate cartilage, bone or ligament repair and formation which mediates associated bone repair.

The chondrogenesis-promoting agent may be used in a method for treating arthritis in a subject. The method may comprise administering to the subject an effective amount of a chondrogenesis-promoting agent. We therefore provide a method for treating arthritis in a subject, comprising administering to the subject chondrogenic cells treated with an effective amount of an chondrogenesis-promoting agent.

We further provide a composition for inducing chondrogenesis and associated skeletal development in a vertebrate, the composition comprising a chondrogenesis-promoting agent and a pharmaceutically acceptable carrier. A morphogenic device for implantation at a cartilage, bone or ligament site in a vertebrate is also provided, the device comprising an implantable biocompatible carrier and a chondrogenesis-promoting agent dispersed within or on said carrier. We describe the use of a composition comprising a chondrogenesis-promoting agent and a pharmaceutically acceptable carrier, for inducing chondrogenesis in vitro.

A chondrocyte may be produced from a chondroprogenitor mesenchymal cell by contacting a chondroprogenitor mesenchymal cell with a chondrogenesis-promoting agent in vitro. The term “chondrocyte” refers to a cell (such as a cartilage, bone or ligament cell) that gives rise to normal cartilage tissue growth in vivo; these cells synthesize and deposit the supportive matrix (composed principally of collagen and proteoglycan) of cartilage.

We provide an implantable prosthetic device for repairing cartilage, bone or ligament associated orthopedic defects, injuries or anomalies in a vertebrate, the device comprising: a prosthetic implant having a surface region implantable adjacent to or within a cartilage, bone or ligament tissue. a chondrogenesis-promoting agent composition disposed on the surface region in an amount sufficient to promote enhanced cartilage, bone or ligament growth into the surface. A method for promoting in vivo integration of an implantable prosthetic device into a target cartilage, bone or ligament tissue of a vertebrate is also described, the method comprising the steps of: providing on a surface of the prosthetic device a composition comprising a chondrogenesis-promoting agent and a pharmaceutically acceptable carrier and implanting the device in a vertebrate at a site where the target cartilage, bone or ligament tissue and the surface of the prosthetic device are maintained at least partially in contact for a time sufficient to permit tissue growth between the target cartilage, bone or ligament tissue and the device.

We provide a method for promoting natural bone formation at a site of skeletal surgery in a vertebrate, the method comprising the steps of delivering a chondrogenesis-promoting agent composition to the site of the skeletal surgery whereby such delivery indirectly promotes the formation of new bone tissue mediated by cartilage.

We describe a method for repairing large segmental skeletal gaps and non-union fractures arising from trauma or surgery in a vertebrate, the method comprising delivering a chondrogenesis-promoting agent composition as described here to the site of the segmental skeletal gap or non-union fracture whereby such delivery promotes the formation of cartilage which mediates new bone tissue formation.

We provide a method for aiding the attachment of an implantable prosthesis to a cartilage, bone or ligament site and for maintaining the long term stability of the prosthesis in a vertebrate, the method comprising coating selected regions of an implantable prosthesis with a chondrogenesis-promoting agent composition and implanting the coated prosthesis into the cartilage, bone or ligament site, whereby such implantation promotes the formation of new cartilage, bone or ligament tissue and indirectly stimulates bone formation.

We provide a method of producing cartilage, bone or ligament at a cartilage, bone or ligament defect site in vivo, the method comprising: implanting into the defect site a population of chondrogenic cells which have been cultured in vitro in the presence of a chondrogenesis-promoting agent as described here.

We provide a method for treating a degenerative joint disease characterized by cartilage, bone or ligament degeneration, the method comprising: delivering a therapeutically effective amount of a chondrogenesis-promoting agent as described here to a disease site.

A pharmaceutical composition comprising at least one chondrogenesis-promoting agent as described here may be applied locally to a treatment site, for example by means of a biodegradable sponge, gel, coating or paste. A suitable gel for use would be a collagen type gel such as collagen I. The chondrogenesis-promoting agent may also be used for the treatment of orthopedic or dental implants to enhance or accelerate osseous integration. A pharmaceutical composition comprising at least one chondrogenesis-promoting agent may be directly applied locally to the site of desired osseous integration or alternatively as a coating on implants.

The chondrogenesis-promoting agent may also be used for promoting in vivo integration of implantable prosthetic devices. In general, the chondrogenesis-promoting agent compositions described here may be applied to synthetic bone grafts for implantation whereby the composition stimulates cartilage, bone or ligament formation and indirectly bone formation. The compositions thus have numerous applications in the orthopedic industry. In particular, there are applications in the fields of trauma repair, spinal fusion, reconstructive surgery, maxillo-facial surgery and dental surgery. The ability of the chondrogenesis-promoting agent compositions to stimulate local natural bone growth provides stability and rapid integration, while the body's normal cell-based bone remodeling process slowly resorbs and replaces a selected implant with natural bone. Implants suitable for in vivo use are generally known to those skilled in the art.

The chondrogenesis-promoting agents described here may be used for cartilage, bone or ligament and skeletal reconstruction. In such an application, the chondrogenesis-promoting agents can be used for ex vivo tissue engineering of cartilage, bone or ligament or skeletal tissue for implantation in a vertebrate. Cells can be treated with a chondrogenesis-promoting agent during osteochondral autograft or allograft transplantations (Minas et al. (1997) Orthopedics 20, 525 538). In autograft transplantations, chondrogenic cells or cells with chondrogenic potential are removed from a patient (e.g. from a rib) and used to fill a cartilaginous lesion. An alternative method involves expanding these cells in vitro, then implanting them into a cartilaginous lesion. A pharmaceutical composition comprising at least one chondrogenesis-stimulating chondrogenesis-promoting agent may be used to treat the cells in in vitro culture prior to engraftment and/or after engraftment through intra-articular injection. The use of the chondrogenesis-promoting agent compositions described here may eliminate the pain and costs associated with the bone harvest procedure required in autograft transplants. Furthermore, the chondrogenesis-promoting agent compositions can be made synthetically thus reducing the possibility of transmission of infection and disease, as well as diminishing the likelihood of immunological rejection by the patient.

The chondrogenesis-promoting agent compositions described here may also be used for the treatment of arthritis, either osteoarthritis or other types of arthritis including rheumatoid arthritis. To reverse or slow degenerative joint disease characterized by cartilage, bone or ligament degeneration, a pharmaceutical composition comprising at least one chondrogenesis-stimulating chondrogenesis-promoting agent may be applied locally through intra-articular injection or in combination with a viscosupplement. The composition may be provided in either a fast-release or slow-release formulation. Such compositions have use in patients with degenerative hip or knee joints, for example.

In general, the chondrogenesis-promoting agents may be used to stimulate in vitro chondrogenesis from mesenchymal precursor cells and in vitro formation of chondrocytes. Such cell culture materials and methods are known to those skilled in the art. Cells and tissues treated with a selected chondrogenesis-promoting agent in vitro can be used therapeutically in vivo or alternatively for in vitro cellular assay systems.

For example, chondrocyte expansion in vivo or in vitro may be for cartilage, bone or ligament repair. Chondrogenic progenitor cells such as mesenchymal stem cells may be removed from, for example, a bone marrow sample from an individual. The isolated chondrogenic progenitor cells such as mesenchymal stem cells may be cultured and transfected with a ZNF145 expression vector, such as a lentiviral vector. They may be immortalised by transformation with a telomerase expression vector or a viral protein such as HPV E6, E7 or Bmi-1. A cell line may be derived from such immortalised chondrogenic progenitor cells such as mesenchymal stem cells.

The ZNF145 over-expressing chondrogenic progenitor cells such as mesenchymal stem cells may then be administered into the body of a patient. Prior to this, they may be induced to chondrocyte differentiation, such as by a pellet culture system as described in Liu et al., 2007.

Dissociated cells isolated by the described process may be grown without scaffold support to create a three-dimensional tissue for cartilage repair (U.S. Pat. No. 6,235,316). However, cells expanded via this method can be implanted in combination with suitable biodegradable, polymeric matrix or hydrogel to form new cartilage tissue. Various matrices may be used, including a polymeric hydrogel formed of a material, such as fibrin or alginate, having cells suspended therein, and a fibrous matrix having an interstitial spacing between about 40 and 200 microns. An example polymeric matrix may degrade in about one to two months after implantation; such as polylactic acid-glycolic acid copolymers (U.S. Pat. No. 5,716,404). The matrices can be seeded prior to implantation or implanted, allowed to vascularize, then seeded with cells. (Cima et al., 1991; Vacanti et al., 1988; and Vacanti et al., 1988). Other materials, such as bioactive molecules that enhance vascularization of the implanted tissue and/or inhibit fibrotic tissue ingrowth can be implanted with the matrix to enhance development of more normal tissue.

The pharmaceutical compositions described here may be used in combination with other chondrogenic stimulators, e.g. bone morphogenetic proteins (BMPs) especially BMP-2 and BMP-4, osteogenic proteins (OPs) such as OP-1 and/or cytokines to enhance and/or maintain the effects of the compositions. Both BMPs and OPs are proteins belonging to the TGF-beta superfamily which represent proteins involved in growth and differentiation as well as tissue morphogenesis and repair. It is also understood that the chondrogenesis-promoting agent compositions described here may additionally comprise other chondroinductive agents or factors, defined as any natural or synthetic organic or inorganic chemical or biochemical compound, or mixture of compounds which stimulate chondrogenesis. It is further understood that the chondrogenesis-promoting agent compositions described here may also comprise other growth factors known to have a stimulatory effect on cartilage, bone or ligament growth and formation.

Chondrogenic Progenitor Stem Cells

We provide for the use of ZNF145 over-expressing cells. Such cells may comprise chondrogenic progenitor stem cells. Such cells may be isolated from placenta (Kogler et al., 2004). They may comprise bone marrow mesenchymal stromal cells (Mackay et al., 1998; Kavalkovick et al., 2002), adipose stromal cells (Huang et al., 2004), synovium (DeBari et al., 2004) and periosteum (DeBari et al., 2001).

Mesenchymal Stem Cells

The ZNF145 over-expressing cells may comprise mesenchymal stem cells. Mesenchymal stem cells and their uses are described in Barry F P, Murphy J M. Mesenchymal stem cells: clinical applications and biological characterization. Int J Biochem Cell Biol. 2004; 36:568-584

### ZNF145

The methods and compositions described here make use of ZNF145. ZNF145 is also known as promyelocytic leukemia zinc finger protein, PLZF, Kruppel-like zinc finger protein, zinc finger protein 145 (Kruppel-like, expressed in promyelocytic leukemia). It is described in the Entrez Gene database as GeneID: 7704.

ZNF145 is a member of the Krueppel C2H2-type zinc-finger protein family and encodes a zinc finger transcription factor that contains nine Kruppel-type zinc finger domains at the carboxyl terminus. ZNF145 is located in the nucleus, is involved in cell cycle progression, and interacts with a histone deacetylase. Specific instances of aberrant gene rearrangement at this locus have been associated with acute promyelocytic leukemia (APL). Alternate transcriptional splice variants have been characterized.

A example of a nucleotide sequence of ZNF145 is NM_{—}006006.4. Another nucleic acid sequence of ZNF145 is NM_{—}001018011. These two sequences represent human variants (1) and (2), both of which are encompassed under the term ZNF145 as used in this document. The term ZNF145 also includes homologues, derivatives, fragments and variants of such sequences.

Nucleic acid variants and homologues of ZNF145 include GenBank Accession numbers AF060568.1, AF076613.1, AF076615.1, AF076616.1, AP000908.4 (109922..149132), AP002518.3, AP002755.2 (2007..109322), CH471065.1, 560093.1, AB208916.1, AK126422.1, BC026902.1, BC029812.1, BM969145.1, BX648973.1, Z19002.1, EU446725.1, CCDS8367.1. Unless the context dictates otherwise, each of these sequences, as well as their homologues, variants, derivatives and fragments are encompassed under the term “ZNF145”.

A protein sequence of ZNF145 is NP_{—}005997.2. A further polypeptide sequence of ZNF145 is NP_{—}001018011.1. Both sequences may be described as “ZNF145” as the term is used in this document.

Polypeptide variants and homologues of ZNF145 include AAD03619.1, AAC32847.1, AAC32848.1, AAC32849.1, EAW67241.1, EAW67242.1, AAC60590.2, BAD92153.1, AAH26902.1, AAH29812.1, CAA79489.1, ABZ92254.1, Q05516.2, Q59H43, Q71UL5, Q71UL6, Q71UL7. Unless the context dictates otherwise, each of these sequences, as well as their homologues, variants, derivatives and fragments are encompassed under the term “ZNF145”.

ZNF145 sequences may comprise any one or more functions of native or wild-type ZNF145. Functions of ZNF145 include DNA binding, metal ion binding, protein homodimerization activity, specific transcriptional repressor activity and zinc ion binding. Assays for each of these activities are well known in the art. Processes in which ZNF145 is involved include apoptosis, central nervous system development, mesonephros development, negative regulation of myeloid cell differentiation, negative regulation of transcription, DNA-dependent, transcription and ubiquitin cycle.

Methods of testing whether a particular protein is involved in any of these processes are known in the art, and are specifically described in Bernardo et al., 2007, 359(2):317-322. Cook et al., Proc Natl Acad Sci USA, 1995, 92(6):2249-2253; Shaknovich et al., Mol Cell Biol 1998, 18: 5533-5545; Petrie et al., Oncogene, 2008 May 26.

The terms “ZNF145” and “ZNF145 sequence”, as they are used in this document, should be taken to include reference to each of the above sequences, as well as to their fragments, homologues, derivatives and variants. ZNF145 nucleic acids, ZNF145 polypeptides, as well as fragments, homologues, derivatives and variants thereof are described in further detail elsewhere in this document.

### ZNF145 Properties

The following text is adapted from OMIM entry 176797: Zinc Finger- and Btb Domain-Containing Protein 16; ZBTB16, also known as Zinc Finger Protein 145; Znf145, Promyelocytic Leukemia Zinc Finger; Plzf, Plzf/Rara Fusion Gene, Included.

Chen et al. (1993, J. Clin. Invest. 91: 2260-2267, 1993) identified the PLZF gene on chromosome 11 as the fusion partner of the retinoic acid receptor-alpha gene (RARA; 180240) on chromosome 17 in a Chinese patient with acute promyelocytic leukemia (APL) and a translocation t(11;17)(q23;21). Chen et al. (1993, EMBO J. 12: 1161-1167, 1993) described the PLZF gene.

Reid et al. (1995, Blood 86: 4544-4552, 1995) showed that murine PLZF is expressed at highest levels in undifferentiated, multipotential hematopoietic progenitor cells and its expression declines as cells become more mature and committed to various hematopoietic lineages. In the human there is a lack of PLZF protein expression in mature peripheral blood mononuclear cells and high PLZF levels in the nuclei of CD34+ human bone marrow progenitor cells. Unlike many transcription factors, PLZF protein in these cells shows a distinct punctate distribution, suggesting its compartmentalization in the nucleus.

Zhang et al. (1999, Proc. Nat. Acad. Sci. 96: 11422-11427, 1999) identified at least 4 alternative splicings (AS-I, -II, -III, and -IV) within exon 1 of the PLZF gene. AS-I was detected in most tissues tested, whereas AS-II, -III, and -IV were present in the stomach, testis, and heart, respectively. Although splicing donor and acceptor signals at exon-intron boundaries for AS-I and exons 1-6 were classic (gt-ag), AS-II, -III, and -IV had atypical splicing sites. These alternative splicings, nevertheless, maintained the open reading frame and may encode isoforms with absence of important functional domains. In mRNA species without AS-I, there is a relatively long 5-prime UTR of 6.0 kb. Zhang et al. (1999, supra) determined that PLZF is a well-conserved gene from C. elegans to human. PLZF paralogous sequences are found in the human genome. The presence of 2 MLL/PLZF-like alignments on human chromosomes 11q23 and 19 suggests a syntenic replication during evolution.

Gene Function

Kang et al. (2003, J. Biol. Chem. 278: 51479-51483, 2003) found that endogenous PLZF in a human promyelocytic cell line was modified by conjugation with SUMO1 (601912) and that PLZF colocalized with SUMO1 in the nucleus of transfected human embryonic kidney cells. Site-directed mutagenesis identified lys242 in transcriptional repression domain-2 as the site of PLZF sumoylation. Reporter gene assays suggested that SUMO1 modification of lys242 was required for transcriptional repression by PLZF, and electrophoretic mobility shift assays showed sumoylation increased the DNA-binding activity of PLZF. PLZF-mediated regulation of the cell cycle and transcriptional repression of the cyclin A2 gene (CCNA2; 123835) were also dependent on sumoylation of PLZF on lys242.

Ikeda et al. (2005, J. Biol. Chem. 280: 8523-8530, 2005) found that PLZF was 1 of 24 genes upregulated during osteoblastic differentiation of cultured OPLL (602475) ligament cells. PLZF was highly expressed during osteoblastic differentiation in all ligament and mesenchymal stem cells examined. Silencing of the PLZF gene by small interfering RNA in human and mouse mesenchymal stem cells reduced expression of osteoblast-specific genes, such as alkaline phosphatase (ALPL; 171760), collagen 1A1 (COL1A1; 120150), Cbfa1 (RUNX2; 600211), and osteocalcin (BGLAP; 112260). PLZF expression was unaffected by the addition of BMP2 (112261), and BMP2 expression was not affected by PLZF expression. In a mouse mesenchymal cell line, overexpression of PLZF increased expression of Cbfa1 and Col1a1; on the other hand, CBFA1 overexpression did not affect expression of Plzf. Ikeda et al. (2005, supra) concluded that PLZF plays a role in early osteoblastic differentiation and is an upstream regulator of CBFA1.

Using yeast 2-hybrid analysis and protein pull-down assays, Rho et al. (2006, FEBS Lett. 580: 4073-4080, 2006) showed that PLZF interacted with the CCS3 isoform of EEF1A1 (130590). Mutation analysis revealed that repressor domain-2 and the zinc finger domain of PLZF were required for the interaction. CCS3 was required for the transcriptional effects of PLZF in reporter gene assays.

Tissing et al. (2007, Blood 109: 3929-3935, 2007) found that 8 hours of prednisolone treatment altered expression of 51 genes in leukemic cells from children with precursor-B- or T-acute lymphoblastic leukemia compared with nonexposed cells. The 3 most highly upregulated genes were FKBP5 (602623), ZBTB16, and TXNIP (606599), which were upregulated 35.4-, 8.8-, and 3.7-fold, respectively.

PLZF/RARA Fusion Protein

Chen et al. (1994, Proc. Nat. Acad. Sci. 91: 1178-1182, 1994) cloned cDNAs encoding PLZF-RARA chimeric proteins and studied their transactivating activities. A ‘dominant-negative’ effect was observed when PLZF-RARA fusion proteins were cotransfected with vectors expressing RARA and retinoid X receptor alpha (RXRA; 180245). These abnormal transactivation properties observed in retinoic acid-sensitive myeloid cells strongly implicated the fusion proteins in the molecular pathogenesis of APL.

Lin et al. (1998, Nature 391: 811-814, 1998) reported that the association of PLZF-RAR-alpha and PML-RAR-alpha (see 102578) with the histone deacetylase complex (see 605164) helps to determine both the development of APL and the ability of patients to respond to retinoids. Consistent with these observations, inhibitors of histone deacetylase dramatically potentiate retinoid-induced differentiation of retinoic acid-sensitive, and restore retinoid responses of retinoic acid-resistant, APL cell lines. Lin et al. (1998) concluded that oncogenic retinoic acid receptors mediate leukemogenesis through aberrant chromatin acetylation, and that pharmacologic manipulation of nuclear receptor cofactors may be a useful approach in the treatment of human disease.

Grignani et al. (1998, Nature 391: 815-818, 1998) demonstrated that both PML-RAR-alpha and PLZF-RAR-alpha fusion proteins recruit the nuclear corepressor (NCOR; see 600849)-histone deacetylase complex through the RAR-alpha CoR box. PLZF-RAR-alpha contains a second, retinoic acid-resistant binding site in the PLZF amino-terminal region. High doses of retinoic acid release histone deacetylase activity from PML-RAR-alpha, but not from PLZF-RAR-alpha. Mutation of the NCOR binding site abolishes the ability of PML-RAR-alpha to block differentiation, whereas inhibition of histone deacetylase activity switches the transcriptional and biologic effects of PLZF-RAR-alpha from being an inhibitor to an activator of the retinoic acid signaling pathway. Therefore, Grignani et al. (1998, supra) concluded that recruitment of histone deacetylase is crucial to the transforming potential of APL fusion proteins, and the different effects of retinoic acid on the stability of the PML-RAR-alpha and PLZF-RAR-alpha corepressor complexes determines the differential response of APLs to retinoic acid.

Guidez et al. (2007, Proc. Nat. Acad. Sci. 104: 18694-18699, 2007) identified CRABP1 (180230) as a target of both PLZF and the RARA/PLZF fusion protein. PLZF repressed CRABP1 through propagation of chromatin condensation from a remote intronic binding element, culminating in silencing of the CRABP1 promoter. Although the canonical PLZF/RARA oncoprotein had no effect on PLZF-mediated repression, the reciprocal translocation product, RARA/PLZF, bound to this remote binding site, recruited p300 (EP300; 602700), and induced promoter hypomethylation and CRABP1 upregulation. Similarly, retinoic acid-resistant murine blasts that expressed both fusion proteins expressed much higher levels of Crabp1 than retinoic acid-sensitive cells expressing Plzf/Rara alone. RARA/PLZF conferred retinoic acid resistance to a retinoid-sensitive acute myeloid leukemia cell line in a CRABP1-dependent fashion. Guidez et al. (2007) concluded that upregulation of CRABP1 by RARA/PLZF contributes to retinoid resistance in leukemia.

Biochemical Features

Ahmad et al. (1998, Proc. Nat. Acad. Sci. 95: 12123-12128, 1998) reported the crystal structure of the BTB domain of PLZF. The BTB domain (also known as the POZ domain) is an evolutionarily conserved protein-protein interaction motif found at the N terminus of 5 to 10% of C2H2-type zinc finger transcription factors. The BTB domain has transcriptional repression activity and interacts with components of the histone deacetylase complex. The latter association provides a mechanism of linking the transcription factor with enzymatic activities that regulate chromatin conformation.

Gene Structure

Zhang et al. (1999, Proc. Nat. Acad. Sci. 96: 11422-11427, 1999) sequenced a 201-kb genomic DNA region containing the entire PLZF gene. Repeated elements accounted for 19.83%, and no obvious coding information other than PLZF was present in this region. PLZF was found to contain 6 exons and 5 introns, and the exon organization corresponded well with protein domains. Zhang et al. (1999, supra) identified at least 4 alternative splicings (AS-I, -II, -III, and -IV) within exon 1.

Van Schothorst et al. (1999, Gene 236: 21-24, 1999) determined that the ZNF145 gene contains 7 exons and spans at least 120 kb. The untranslated exon 1 is located within a CpG island, and several SP1 (189906)- and GATA1 (305371)-binding sites are upstream of exon 1.

Mapping

By FISH, Chen et al. (1993, J. Clin. Invest. 91: 2260-2267, 1993) localized the PLZF gene to chromosome 11q23.1.

Cytogenetics

Almost all patients with APL have a chromosomal translocation t(15;17)(q22;q21). Molecular studies reveal that the translocation results in a chimeric gene through fusion between the promyelocytic leukemia gene (PML; 102578) on chromosome 15 and the retinoic acid receptor-alpha gene (RARA; 180240) on chromosome 17. Chen et al. (1993, J. Clin. Invest. 91: 2260-2267, 1993) reported studies of a Chinese patient with APL and a variant translocation t(11;17)(q23;21) in which the PLZF gene on chromosome 11q23.1 was fused to the RARA gene on chromosome 17. Similar to t(15;17) APL, all-trans retinoic acid treatment produced an early leukocytosis which was followed by a myeloid maturation, but the patient died too early to achieve remission.

Zhang et al. (1999, Proc. Nat. Acad. Sci. 96: 11422-11427, 1999) characterized the chromosomal breakpoints and joining sites in the index acute promyelocytic leukemia case with t(11;17), reported by Chen et al. (1993). The results suggested the involvement of a DNA damage-repair mechanism.

Animal Model

Cheng et al. (1999, Proc. Nat. Acad. Sci. 96: 6318-6323, 1999) generated transgenic mice with PLZF-RARA and NPM (164040)-RARA. PLZF-RARA transgenic animals developed chronic myeloid leukemia-like phenotypes at an early stage in life (within 3 months in 5 of 6 mice), whereas 3 NPM-RARA transgenic mice showed a spectrum of phenotypes from typical APL to chronic myeloid leukemia relatively late in life (from 12 to 15 months). In contrast to bone marrow cells from PLZF-RARA transgenic mice, those from NPM-RARA transgenic mice could be induced to differentiate by all-trans-retinoic acid (ATRA). Cheng et al. (1999, supra) found that in interacting with nuclear coreceptors the 2 fusion proteins had different ligand sensitivities, which may be the underlying molecular mechanism for differential responses to ATRA. These data clearly established the leukemogenic role of PLZF-RARA and NPM-RARA and the importance of fusion receptor/corepressor interactions in the pathogenesis as well as in determining different clinical phenotypes of APL.

He et al. (2000, Molec. Cell 6: 1131-1141, 2000) generated transgenic mice expressing RARA-PLZF and PLZF-RARA in their promyelocytes. RARA-PLZF transgenic mice did not develop leukemia. However, PLZF-RARA/RARA-PLZF double transgenic mice developed leukemia with classic APL features. The authors demonstrated that RARA-PLZF can interfere with PLZF transcriptional repression, and that this is critical for APL pathogenesis, since leukemias in PLZF-deficient/PLZF-RARA mutants and in PLZF-RARA/RARA-PLZF transgenic mice were indistinguishable. Thus, both products of a cancer-associated translocation are crucial in determining the distinctive features of the disease.

Barna et al. (2000, Nature Genet. 25: 166-172, 2000) generated Zfp145−/− mice and showed that Plzf is essential for patterning of the limb and axial skeleton. Inactivation of the gene resulted in patterning defects affecting all skeletal structures of the limb, including homeotic transformations of anterior skeletal elements into posterior structures. They demonstrated that Plzf acts as a growth-inhibitory and proapoptotic factor in the limb bud. The expression of members of the Abdominal B (Abdb) Hox gene complex (see 142956), as well as genes encoding bone morphogenetic proteins (e.g., 112267), was altered in the developing limb of the Zfp145−/− mice. The mice also exhibited anterior-directed homeotic transformation throughout the axial skeleton with associated alterations in Hox gene expression. Plzf is, therefore, a mediator of anterior-to-posterior patterning in both the axial and appendicular skeleton and acts as a regulator of Hox gene expression.

Barna et al. (2002, Dev. Cell 3: 499-510, 2002) determined that the defects in Plzf−/− mice were due to spatial, but not temporal, deregulation of the Abdb Hoxd complex. They identified several Plzf-binding sites in Hoxd11 (142986) and showed that Plzf bound Hoxd11 genomic DNA fragments as a dimer or possibly a trimer, mostly when DNA loops were formed. Barna et al. (2002) also found evidence of long-range interactions between distant Plzf-binding sites within the Hoxd regulatory elements. Plzf mediated transcriptional repression of a Hoxd reporter construct, and in the absence of Plzf, there were increased acetylated histones on Hoxd regulatory regions. Plzf showed dose-dependent transcriptional repression of a Hoxd reporter in mouse anterior limb micromass cultures, but there was no repression in posterior limb micromass cultures. Plzf also directly tethered the polycomb protein Bmi1 (164831) on DNA, which antagonized posteriorizing signals in the limb. Barna et al. (2002) concluded that recruitment of histone deacetylases and polycomb proteins by PLZF favors transition from euchromatin to heterochromatin.

Adult germline stem cells are capable of self-renewal, tissue regeneration, and production of large numbers of differentiated progeny. The mouse mutant ‘luxoid’ (lu) arose spontaneously and was mapped to mouse chromosome 9 (Green, 1955, J. Hered. 46: 91-99, 1955), and was initially characterized by its semidominant abnormalities and recessive skeletal and male infertility phenotypes (Forsthoefel, 1958, J. Morphol. 102: 247-287, 1958). Buaas et al. (2004, Nature Genet. 36: 647-652, 2004) showed that the mouse mutant luxoid affects adult germline stem cell self-renewal. Young homozygous luxoid mutant mice produce limited numbers of normal spermatozoa and then progressively lose their germline after birth. Transplantation studies showed that germ cells of mutant mice did not colonize recipient testes, suggesting that the defect is intrinsic to the stem cells. Buaas et al. (2004) determined that the luxoid mutant contains a nonsense mutation in the Plzf gene, a transcriptional repressor that regulates the epigenetic state of undifferentiated cells. They showed, furthermore, that Plzf is coexpressed with Oct4 (164177) in undifferentiated spermatogonia. This was said to be the first gene found to be required in germ cells for stem cell self-renewal in mammals.

Costoya et al. (2004, Nature Genet. 36: 653-659, 2004) likewise showed that Plzf has a crucial role in spermatogenesis. Expression of the gene was restricted to gonocytes and undifferentiated spermatogonia and was absent in the tubules of W/W(v) mutants that lack these cells. Mice lacking Plzf underwent a progressive loss of spermatogonia with age, associated with increases in apoptosis and subsequent loss of tubule structure but without overt differentiation defects or loss of the supporting Sertoli cells. Spermatogonia transplantation experiments revealed a depletion of spermatogonia stem cells in the adult. These and other results identified Plzf as a spermatogonia-specific transcription factor in the testis that is required to regulate self-renewal and maintenance of the stem cell pool.

Barna et al. (2005, Nature 436: 277-281, 2005) identified a genetic interaction between Gli3 (165240) and Plzf that is required specifically at very early stages of limb development for all proximal cartilage condensations in the hindlimb (femur, tibia, fibula). Notably, distal condensations comprising the foot were relatively unperturbed in Gli3/Plzf double knockout mouse embryos. Barna et al. (2005, supra) demonstrated that the cooperative activity of Gli3 and Plzf establishes the correct temporal and spatial distribution of chondrocyte progenitors in the proximal limb bud independently of proximal-distal (P-D) patterning markers and overall limb bud size. Moreover, the limb defects in the double knockout embryos correlated with the transient death of a specific subset of proximal mesenchymal cells that express bone morphogenetic protein receptor type 1B (Bmpr1b; 603248) at the onset of limb development. Barna et al. (2005, supra) concluded that development of proximal and distal skeletal elements is distinctly regulated during early limb bud formation. The initial division of the vertebrate limb into 2 distinct molecular domains is consistent with fossil evidence indicating that the upper and lower extremities of the limb have different evolutionary origins.

### ZNF145 Polypeptides

The methods and compositions described here make use of ZNF145 polypeptides, which are described in detail below.

As used here, the term “ZNF145 polypeptide” is intended to refer to a sequence having GenBank Accession number NP_{—}005997.2 (protein variant 1) or NP_{—}001018011.1 (protein variant 2). Other ZNF145 polypeptide sequences include AAD03619.1, AAC32847.1, AAC32848.1, AAC32849.1, EAW67241.1, EAW67242.1, AAC60590.2, BAD92153.1, AAH26902.1, AAH29812.1, CAA79489.1, ABZ92254.1, Q05516.2, Q59H43, Q71UL5, Q71UL6 and Q71UL7.

A “ZNF145 polypeptide” may comprise or consist of a human ZNF145 polypeptide, such as the sequence having accession number NP_{—}005997.2 or NP_{—}001018011.1.

Homologues variants and derivatives thereof of any, some or all of these polypeptides are also included. For example, ZNF145 may include GenBank Accession Number AAD03619.

ZNF145 polypeptides may be used for a variety of means, for example, administration to an individual suffering from, or suspected to be suffering from, a degenerative disease, for the treatment thereof They may also be used for production or screening of anti-ZNF145 agents such as specific ZNF145 binding agents, in particular, anti-ZNF145 antibodies. These are described in further detail below. The expression of ZNF145 polypeptides may be detected for diagnosis or detection of a degenerative disease.

A “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed:, Academic Press, New York, 1983; Seifter et al., “Analysis for protein modifications and nonprotein cofactors”, Meth Enzymol (1990) 182:626-646 and Rattan et aL, “Protein Synthesis: Posttranslational Modifications and Aging”, Ann NY Acad Sci (1992) 663:48-62.

The term “polypeptide” includes the various synthetic peptide variations known in the art, such as a retroinverso D peptides. The peptide may be an antigenic determinant and/or a T-cell epitope. The peptide may be immunogenic in vivo. The peptide may be capable of inducing neutralising antibodies in vivo.

As applied to ZNF145, the resultant amino acid sequence may have one or more activities, such as biological activities in common with a ZNF145 polypeptide, for example a human ZNF145 polypeptide. For example, a ZNF145 homologue may have a increased expression level in a chondrogenic mesenchymal stem cell compared to a non-chondrogenic mesenchymal stem cell. In particular, the term “homologue” covers identity with respect to structure and/or function providing the resultant amino acid sequence has ZNF145 activity. With respect to sequence identity (i.e. similarity), there may be at least 70%, such as at least 75%, such as at least 85%, such as at least 90% sequence identity. There may be at least 95%, such as at least 98%, sequence identity. These terms also encompass polypeptides derived from amino acids which are allelic variations of the ZNF145 nucleic acid sequence.

Where reference is made to the “activity” or “biological activity” of a polypeptide such as ZNF145, these terms are intended to refer to the metabolic or physiological function of ZNF145, including similar activities or improved activities or these activities with decreased undesirable side effects. Also included are antigenic and immunogenic activities of the ZNF145. Examples of such activities, and methods of assaying and quantifying these activities, are known in the art, and are described in detail elsewhere in this document.

For example, such activities may include any one or more of the following: DNA binding, metal ion binding, protein homodimerization activity, specific transcriptional repressor activity and zinc ion binding, apoptosis, central nervous system development, mesonephros development, negative regulation of myeloid cell differentiation, negative regulation of transcription, DNA-dependent, transcription and ubiquitin cycle. Methods of testing whether a particular protein is involved in any of these processes are also known in the art.

Other ZNF145 Polypeptides

ZNF145 variants, homologues, derivatives and fragments are also of use in the methods and compositions described here.

The terms “variant”, “homologue”, “derivative” or “fragment” in relation to ZNF145 include any substitution of, variation of, modification of, replacement of, deletion of or addition of one (or more) amino acid from or to a sequence. Unless the context admits otherwise, references to “ZNF145” includes references to such variants, homologues, derivatives and fragments of ZNF145.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

ZNF145 polypeptides as described here may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

ZNF145 polypeptides may further comprise heterologous amino acid sequences, typically at the N-terminus or C-terminus, such as the N-terminus. Heterologous sequences may include sequences that affect intra or extracellular protein targeting (such as leader sequences). Heterologous sequences may also include sequences that increase the immunogenicity of the ZNF145 polypeptide and/or which facilitate identification, extraction and/or purification of the polypeptides. Another heterologous sequence that may be used is a polyamino acid sequence such as polyhistidine which may be N-terminal. A polyhistidine sequence of at least 10 amino acids, such as at least 17 amino acids but fewer than 50 amino acids may be employed.

The ZNF145 polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

ZNF145 polypeptides as described here are advantageously made by recombinant means, using known techniques. However they may also be made by synthetic means using techniques well known to skilled persons such as solid phase synthesis. Such polypeptides may also be produced as fusion proteins, for example to aid in extraction and purification. Examples of fusion protein partners include glutathione-S-transferase (GST), 6× His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. The fusion protein may be one which does not hinder the function of the protein of interest sequence.

The ZNF145 polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the ZNF145 protein may be mixed with carriers or diluents which will not interfere with the intended purpose of the protein and still be regarded as substantially isolated. A ZNF145 polypeptide may also be in a substantially purified form, in which case it will generally comprise the protein in a preparation in which more than 90%, for example, 95%, 98% or 99% of the protein in the preparation is a ZNF145 polypeptide.

By aligning ZNF145 sequences from different species, it is possible to determine which regions of the amino acid sequence are conserved between different species (“homologous regions”), and which regions vary between the different species (“heterologous regions”).

The ZNF145 polypeptides may therefore comprise a sequence which corresponds to at least part of a homologous region. A homologous region shows a high degree of homology between at least two species. For example, the homologous region may show at least 70%, at least 80%, at least 90% or at least 95% identity at the amino acid level using the tests described above. Peptides which comprise a sequence which corresponds to a homologous region may be used in therapeutic strategies as explained in further detail below. Alternatively, the ZNF145 peptide may comprise a sequence which corresponds to at least part of a heterologous region. A heterologous region shows a low degree of homology between at least two species.

ZNF145 Homologues

The ZNF145 polypeptides disclosed for use include homologous sequences obtained from any source, for example related viral/bacterial proteins, cellular homologues and synthetic peptides, as well as variants or derivatives thereof. Thus polypeptides also include those encoding homologues of ZNF145 from other species including animals such as mammals (e.g. mice, rats or rabbits), especially primates, more especially humans. More specifically, homologues include human homologues.

In the context of this document, a homologous sequence is taken to include an amino acid sequence which is at least 15, 20, 25, 30, 40, 50, 60, 70, 80 or 90% identical, such as at least 95 or 98% identical at the amino acid level, for example over at least 50 or 100, 200, 300, 400 or 500 amino acids with the sequence of a relevant ZNF145 sequence.

In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i.e. amino acid residues having similar chemical properties/functions), in the context of the present document homology may be expressed in terms of sequence identity.

Homology comparisons can be conducted by eye, or more usually, with the aid of readily available sequence comparison programs. These publicly and commercially available computer programs can calculate % identity between two or more sequences.

% identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local identity or similarity.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, the default values may be used when using such software for sequence comparisons. For example when using the GCG Wisconsin Bestfit package (see below) the default gap penalty for amino acid sequences is -12 for a gap and -4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Altschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). The GCG Bestfit program may be used.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). The public default values for the GCG package may be used, or in the case of other software, the default matrix, such as BLOSUM62.

Once the software has produced an optimal alignment, it is possible to calculate % homology, such as % sequence identity. The software typically does this as part of the sequence comparison and generates a numerical result.

The terms “variant” or “derivative” in relation to amino acid sequences includes any substitution of, variation of, modification of, replacement of, deletion of or addition of one (or more) amino acids from or to the sequence providing the resultant amino acid sequence retains substantially the same activity as the unmodified sequence, such as having at least the same activity as the ZNF145 polypeptides.

Polypeptides having the ZNF145 amino acid sequence disclosed here, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here. Amino acid substitutions may include the use of non-naturally occurring analogues, for example to increase blood plasma half-life of a therapeutically administered polypeptide.

ZNF145 Fragments

Polypeptides for use in the methods and compositions described here also include fragments of the full length sequence of any of the ZNF145 polypeptides identified above. Fragments may comprise at least one epitope. Methods of identifying epitopes are well known in the art. Fragments will typically comprise at least 6 amino acids, such as at least 10, 20, 30, 50 or 100 amino acids.

Included are fragments comprising or consisting of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315 or more residues from a relevant ZNF145 amino acid sequence.

We further describe peptides comprising a portion of a ZNF145 polypeptide as described here. Thus, fragments of ZNF145 and its homologues, variants or derivatives are included. The peptides may be between 2 and 200 amino acids, such as between 4 and 40 amino acids in length. The peptide may be derived from a ZNF145 polypeptide as disclosed here, for example by digestion with a suitable enzyme, such as trypsin. Alternatively the peptide, fragment, etc may be made by recombinant means, or synthesised synthetically.

Such a ZNF145 fragment may be used to generate probes to preferentially detect ZNF145 expression, for example, through antibodies generated against such fragments. These antibodies would be expected to bind specifically to ZNF145, and are useful in the methods of detection, diagnosis and treatment disclosed here.

ZNF145 and its fragments, homologues, variants and derivatives, may be made by recombinant means. However they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6× His, GAL4 (DNA binding and/or transcriptional activation domains) and β-galactosidase. It may also be convenient to include a proteolytic cleavage site between the fusion protein partner and the protein sequence of interest to allow removal of fusion protein sequences. The fusion protein may be one which will not hinder the function of the protein of interest sequence. Proteins may also be obtained by purification of cell extracts from animal cells.

The ZNF145 polypeptides, variants, homologues, fragments and derivatives disclosed here may be in a substantially isolated form. It will be understood that such polypeptides may be mixed with carriers or diluents which will not interfere with the intended purpose of the protein and still be regarded as substantially isolated. A ZNF145 variant, homologue, fragment or derivative may also be in a substantially purified form, in which case it will generally comprise the protein in a preparation in which more than 90%, e.g. 95%, 98% or 99% of the protein in the preparation is a protein.

The ZNF145 polypeptides, variants, homologues, fragments and derivatives disclosed here may be labelled with a revealing label. The revealing label may be any suitable label which allows the polypeptide, etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

A ZNF145 polypeptides, variants, homologues, fragments and derivatives disclosed here, optionally labelled, may also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

Antibodies against ZNF145 are known in the art and are commercially available, for example Rabbit anti-Human PML Polyclonal Antibody-a (Catalogue No AI70002A) and Rabbit anti-Human PML Polyclonal Antibody-b (AI70002B) from Anogen, Mississauga, Ontario, Canada).

The ZNF145 polypeptides, variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the ZNF145 polypeptides, variants, homologues, fragments and derivatives disclosed here are expressed may be used in assay systems to identify candidate substances which interfere with or enhance the functions of the polypeptides in the cell.

### ZNF145 Nucleic Acids

The methods and compositions described here may employ, as a means for detecting expression levels of ZNF145, ZNF145 polynucleotides, ZNF145 nucleotides and ZNF145 nucleic acids, as well as variants, homologues, derivatives and fragments of any of these. In addition, we disclose particular ZNF145 fragments useful for the methods of diagnosis described here. The ZNF145 nucleic acids may also be used for the methods of treatment or prophylaxis described.

The terms “ZNF145 polynucleotide”, “ZNF145 nucleotide” and “ZNF145 nucleic acid” may be used interchangeably, and should be understood to specifically include both cDNA and genomic ZNF145 sequences. These terms are also intended to include a nucleic acid sequence capable of encoding a ZNF145 polypeptide and/or a fragment, derivative, homologue or variant of this.

Where reference is made to a ZNF145 nucleic acid, this should be taken as a reference to any member of the ZNF145 family of nucleic acids. Example ZNF145 nucleic acids include NM_{—}006006.4 (mRNA variant 1) and NM_{—}001018011 (mRNA variant 2). Other ZNF145 nucleic acids include GenBank Accession numbers AF060568.1, AF076613.1, AF076615.1, AF076616.1, AP000908.4 (109922..149132), AP002518.3, AP002755.2 (2007..109322), CH471065.1, S60093.1, AB208916.1, AK126422.1, BC026902.1, BC029812.1, BM969145.1, BX648973.1, Z19002.1, EU446725.1 and CCDS8367.1.

Also included are any one or more of the nucleic acid sequences set out as “Other ZNF145 nucleic acid sequences” below.

For example, the ZNF145 nucleic acid may comprise a human ZNF145 sequence having GenBank Accession Number NM_{—}006006.4 or NM_{—}001018011 (mRNA variant 2).

ZNF145 nucleic acids may be used for a variety of means, for example, administration to an individual suffering from, or suspected to be suffering from, a degenerative disease, for the treatment thereof The expression of ZNF145 nucleic acids may be detected for the detection of a chondrogenic mesenchymal stem cell. ZNF145 nucleic acids may also be used for the expression or production of ZNF145 polypeptides.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

The term “nucleotide sequence” may means DNA.

Other Nucleic Acids

We also provide nucleic acids which are fragments, homologues, variants or derivatives of ZNF145 nucleic acids. The terms “variant”, “homologue”, “derivative” or “fragment” in relation to ZNF145 nucleic acid include any substitution of, variation of, modification of, replacement of, deletion of or addition of one (or more) nucleic acids from or to the sequence of a ZNF145 nucleotide sequence. Unless the context admits otherwise, references to “ZNF145” and “ZNF145” include references to such variants, homologues, derivatives and fragments of ZNF145.

The resultant nucleotide sequence may encode a polypeptide having any one or more ZNF145 activity. The term “homologue” may be intended to cover identity with respect to structure and/or function such that the resultant nucleotide sequence encodes a polypeptide which has ZNF145 activity. For example, a homologue etc of ZNF145 may have a enhanced expression level in chondrogenic mesenchymal stem cells compared to non-chondrogenic mesenchymal stem cells. With respect to sequence identity (i.e. similarity), there may be at least 70%, at least 75%, at least 85% or at least 90% sequence identity. There may be at least 95%, such as at least 98%, sequence identity to a relevant sequence (e.g., a ZNF145 sequence having GenBank accession number NM_{—}015472). These terms also encompass allelic variations of the sequences.

Variants, Derivatives and Homologues

ZNF145 nucleic acid variants, fragments, derivatives and homologues may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Said variant, homologues or derivatives may code for a polypeptide having biological activity. Such fragments, homologues, variants and derivatives of ZNF145 may comprise modulated activity, as set out above.

As indicated above, with respect to sequence identity, a “homologue” may have at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence (e.g., a ZNF145 sequence having GenBank accession number NM_{—}015472).

There may be at least 95% identity, at least 96% identity, at least 97% identity, at least 98% identity or at least 99% identity. Nucleotide identity comparisons may be conducted as described above. A sequence comparison program which may be used is the GCG Wisconsin Bestfit program described above. The default scoring matrix has a match value of 10 for each identical nucleotide and -9 for each mismatch. The default gap creation penalty is -50 and the default gap extension penalty is -3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences may be at least 15 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein (e.g., a ZNF145 sequence having GenBank accession numbe NM_{—}015472). Such polynucleotides may be generally at least 70%, at least 80 or 90% or at least 95% or 98% homologous to the corresponding nucleotide sequences over a region of at least 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P or ³³P or with non-radioactive probes (e.g., fluorescent dyes, biotin or digoxigenin).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

We provide nucleotide sequences that may be able to hybridise to the ZNF145 nucleic acids, fragments, variants, homologues or derivatives under stringent conditions (e.g. 65° C. and 0.1×SSC (1×SSC=0.15 M NaCl, 0.015 M Na₃ Citrate pH 7.0)).

Generation of Homologues, Variants and Derivatives

Polynucleotides which are not 100% identical to the relevant sequences (e.g., a human ZNF145 sequence having GenBank accession number NM_{—}015472) but which are also included, as well as homologues, variants and derivatives of ZNF145 can be obtained in a number of ways. Other variants of the sequences may be obtained for example by probing DNA libraries made from a range of individuals, for example individuals from different populations. For example, ZNF145 homologues may be identified from other individuals, or other species. Further recombinant ZNF145 nucleic acids and polypeptides may be produced by identifying corresponding positions in the homologues, and synthesising or producing the molecule as described elsewhere in this document.

In addition, other viral/bacterial, or cellular homologues of ZNF145, particularly cellular homologues found in mammalian cells (e.g. rat, mouse, bovine and primate cells), may be obtained and such homologues and fragments thereof in general will be capable of selectively hybridising to human ZNF145. Such homologues may be used to design non-human ZNF145 nucleic acids, fragments, variants and homologues. Mutagenesis may be carried out by means known in the art to produce further variety.

Sequences of ZNF145 homologues may be obtained by probing cDNA libraries made from or genomic DNA libraries from other animal species, and probing such libraries with probes comprising all or part of any of the ZNF145 nucleic acids, fragments, variants and homologues, or other fragments of ZNF145 under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the ZNF145 nucleic acids. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the ZNF145 sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, ZNF145 nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, such as at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Primers comprising fragments of ZNF145 are particularly useful in the methods of detection of ZNF145 expression, such as up-regulation of ZNF145 expression, for example, as associated with chondrogenesis. Suitable primers for amplification of ZNF145 may be generated from any suitable stretch of ZNF145. Primers which may be used include those capable of amplifying a sequence of ZNF145 which is specific.

Although ZNF145 primers may be provided on their own, they are most usefully provided as primer pairs, comprising a forward primer and a reverse primer.

Longer polynucleotides will generally be produced using recombinant means, for example using a PCR (polymerase chain reaction) cloning techniques. This will involve making a pair of primers (e.g. of about 15 to 30 nucleotides), bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Polynucleotides or primers may carry a revealing label. Suitable labels include radioisotopes such as ³²P or ³⁵S, digoxigenin, fluorescent dyes, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides, for example, the ZNF145 nucleic acids, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

ZNF145 Control Regions

For some purposes, it may be necessary to utilise or investigate control regions of ZNF145. Such control regions include promoters, enhancers and locus control regions. By a control region we mean a nucleic acid sequence or structure which is capable of modulating the expression of a coding sequence which is operatively linked to it.

For example, control regions are useful in generating transgenic animals expressing ZNF145. Furthermore, control regions may be used to generate expression constructs for ZNF145. This is described in further detail below.

Identification of control regions of ZNF145 is straightforward, and may be carried out in a number of ways. For example, the coding sequence of ZNF145 may be obtained from an organism, by screening a cDNA library using a human or mouse ZNF145 cDNA sequence as a probe. 5′ sequences may be obtained by screening an appropriate genomic library, or by primer extension as known in the art. Database searching of genome databases may also be employed. Such 5′ sequences which are particularly of interest include non-coding regions. The 5′ regions may be examined by eye, or with the aid of computer programs, to identify sequence motifs which indicate the presence of promoter and/or enhancer regions.

Furthermore, sequence alignments may be conducted of ZNF145 nucleic acid sequences from two or more organisms. By aligning ZNF145 sequences from different species, it is possible to determine which regions of the amino acid sequence are conserved between different species. Such conserved regions are likely to contain control regions for the gene in question (i.e., ZNF145). The mouse and human genomic sequences as disclosed here, for example, a mouse ZNF145 genomic sequence, may be employed for this purpose. Furthermore, ZNF145 homologues from other organisms may be obtained using standard methods of screening using appropriate probes generated from the mouse and human ZNF145 sequences. The genome of the pufferfish (Takifugu rubripes) or zebrafish may also be screened to identify a ZNF145 homologue; thus, several zebrafish sequences of ZNF145 have been identified (noted above). Comparison of the 5′ non-coding region of the Fugu or zebrafish ZNF145 gene with a mouse or human genomic ZNF145 sequence may be used to identify conserved regions containing control regions.

Deletion studies may also be conducted to identify promoter and/or enhancer regions for ZNF145.

The identity of putative control regions may be confirmed by molecular biology experiments, in which the candidate sequences are linked to a reporter gene and the expression of the reporter detected.

### ZNF145 Over-Expressing Cells

The methods and compositions described here use, in some aspects, a cell which over-expresses ZNF145, that is to say, a cell with up-regulated expression or activity of ZNF145. Such a cell may comprise a chondrogenic progenitor cell such as a mesenchymal stem cell. It, or an ancestor of it, may be engineered to possess such properties.

In general, ZNF145 over-expressing cells may be constructed by transfecting or otherwise introducing a ZNF145 expression vector (as described below) into a suitable host cell, for example, a chondrogenic progenitor stem cell such as a mesenchymal stem cell.

A cell which over-expresses ZNF145 may display enhanced expression of a chondrogenic marker. The chondrogenic marker may comprise any marker for chondrogenesis as known in the art. For example, the chondrogenic marker may comprise collagen type 2 (COL2A1). Such markers may be detected by methods known in the art, for example using antibodies and histological staining, Western blots, etc. Either of the two Collagen type 2 variants, i.e., Col2A1 variant 1 (GenBank Accession Number NM_{—}001844) and Col2A1 variant 2 (GenBank Accession Number NM_{—}033150) may be detected. Similarly, either of the two aggrecan variants, i.e., Aggrecan variant 1 (GenBank Accession Number NM_{—}001135) and Aggrecan variant 2 (GenBank Accession Number NM_{—}013227) may be detected. Col10A1 (GenBank Accession Number NM_{—}000493) and Sox9 (GenBank Accession Number NM_{—}000346) may also be detected as a chondrogenic marker, by methods known in the art.

A cell which over-expresses ZNF145 may display enhanced secretion of cartilage, bone or ligament proteoglycans. Such enhanced secretion may be detected by methods known in the art, such as detected by alcian blue staining. It may display an improved ability to repair a cartilage, bone or ligament defect. This may be detected by histological grading of any one or more of cell morphology, matrix-staining, surface regularity, thickness of cartilage, bone or ligament and integration of donor with host adjacent cartilage. Histological grading as described by Wakitani et al (1994) may be used in such assays.

The ZNF145 over-expressing cell may be cultured into a cell line. The ZNF145 over-expressing cell may be immortalised by means known in the art, for example, by expression of telomerase, as described in detail in the Examples.

In order to up-regulate the expression of ZNF145, a ZNF145 polynucleotide sequence may be brought into association with a regulatory sequence so as to enable the regulatory sequence to direct expression of the ZNF145 polynucleotide. Expression of the polypeptide under control of the regulatory sequence is then allowed to happen within a suitable target cell, such as a mesenchymal stem cell.

The regulatory sequence may be one with which the ZNF145 polynucleotide sequence is not naturally associated.

We describe a method of expressing a ZNF145 polypeptide comprising providing a cell, such as a mesenchymal stem cell, in which a ZNF145 polynucleotide sequence has been brought into association with a regulatory sequence so as to enable the regulatory sequence to direct expression of said polynucleotide, and culturing the cell under conditions which enable expression of the polypeptide.

We further describe a method of producing a polypeptide comprising: (a) providing an expression sequence produced by bringing a ZNF145 polynucleotide sequence into association with a regulatory sequence so as to enable the regulatory sequence to direct expression of said polynucleotide; and (b) allowing expression of the polypeptide from the expression sequence under control of the regulatory sequence.

In particular, the ZNF145 nucleotide sequences encoding the respective ZNF145 nucleic acid or homologues, variants, or derivatives thereof may be inserted into appropriate expression vector, i.e., a vector which contains the necessary elements for the transcription and translation of the inserted coding sequence.

We also provide for a polypeptide produced by any of the above methods.

Methods of enabling expression of ZNF145 polypeptides are set out below. It will be appreciated that these methods may be suitable for use in embodiments of the methods and compositions described here in which up-regulation of a polypeptide is desired, e.g., up-regulation of ZNF145 in order to achieve enhanced chondrogenesis of a mesenchymal stem cell.

One method by which to provide expressed polypeptides is by means of an expression vector, i.e., a vector (e.g., a plasmid) which contains a regulatable promoter, optionally with other regulatory sequences such as enhancers, which is operably linked to a sequence encoding a polypeptide of interest such as ZNF145 which has been cloned into the expression vector. This is described in further detail below.

Methods which are well known to those skilled in the art may be used to construct expression vectors containing sequences encoding ZNF145 and appropriate transcriptional and translational control elements. These methods include in vitro recombinant DNA techniques, synthetic techniques, and in vivo genetic recombination. Such techniques are described in Sambrook, J. et al. (1989; Molecular Cloning, A Laboratory Manual, ch. 4, 8, and 16-17, Cold Spring Harbor Press, Plainview, N.Y.) and Ausubel, F. M. et al. (1995 and periodic supplements; Current Protocols in Molecular Biology, ch. 9, 13, and 16, John Wiley & Sons, New York, N.Y.).

A variety of expression vector/host systems may be utilized to contain and express sequences encoding ZNF145. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. Any suitable host cell may be employed. Mammalian cell expression is described in further detail below.

The “control elements” or “regulatory sequences” are those non-translated regions of the vector (i.e., enhancers, promoters, and 5′ and 3′ untranslated regions) which interact with host cellular proteins to carry out transcription and translation. Such elements may vary in their strength and specificity. Depending on the vector system and host utilized, any number of suitable transcription and translation elements, including constitutive and inducible promoters, may be used. These are described in further detail below.

The cell over-expressing ZNF145 may be biased towards chondrogenic differentiation. This may be achieved by various means known in the art, for example, by a pellet culture system as described in Liu et al., 2007. The method set out in this reference comprises by pelleting chondrogenic progenitor cells, for example a mesenchymal stem cells, and culturing in chondrogenic medium containing 10 ng/ml transforming growth factor (TGF)-β3, 10-7 M dexamethasone, 50 μg/ml ascorbate-2-phosphate, 40 μg/ml proline, 100 μg/ml pyruvate, and 50 mg/ml ITS+Premix (Becton Dickinson; 6.25 μg/ml insulin, 6.25 μg/ml transferrin, 6.25 μg/ml selenious acid, 1.25 mg/ml BSA, and 5.35 mg/ml linoleic acid). Other methods of biasing cells towards chondrogenic differentiation are known in the art, and may be used in the methods and compositions described here.

In addition to over-expressing ZNF145 in the cell, the expression of other proteins may be controlled to improve or enhance chondrogenesis. For example, the cell (or an ancestor thereof) may be engineered to increase expression or activity of any one or more of the following: Nanog, Oct4, telomerase, SV40 large T antigen, HPV E6, HPV E7 and Bmi-1.

### ZNF145 Expression Vectors

For the purpose of ZNF145 manipulation and over-expression in mesenchymal stem cells, ZNF145 polynucleotides may be incorporated into a recombinant replicable vector. The vector may be used to replicate the nucleic acid in a compatible host cell.

We provide a method of making polynucleotides by introducing a polynucleotide into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

ZNF145 polynucleotides may also be incorporated into an expression vector, for expression of ZNF145 in a mesenchymal stem cell. For example, a ZNF145 polynucleotide in a vector may be operably linked to a control sequence that is capable of providing for the expression of the coding sequence by the host cell, i.e. the vector is an expression vector. The term “operably linked” means that the components described are in a relationship permitting them to function in their intended manner. A regulatory sequence or regulatory region “operably linked” to a coding sequence is ligated in such a way that expression of the coding sequence is achieved under condition compatible with the control sequences.

Regulatory Regions

In a preferred embodiment, ZNF145 is expressed in a mesenchymal stem cell by being operatively linked to a regulatory region of a gene expressed by such a cell.

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

Control sequences for use here may be modified, for example by the addition of further transcriptional regulatory elements to make the level of transcription directed by the control sequences more responsive to transcriptional modulators.

The term promoter is used here to refer to a group of transcriptional control modules that are clustered around the initiation site for RNA polymerase II. Much of the thinking about how promoters are organized derives from analyses'of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV 40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way. Accordingly, a ZNF145 expression vector may comprise a promoter, an enhancer, or both, in addition to ZNF145 coding sequences.

Viral Transformation of MSCS with Expression Constructs for ZNF145

ZNF145 vectors, such as ZNF145 expression vectors, may be transformed or transfected into a suitable host cell such as an mesenchymal stem cell as described below to provide for expression of ZNF145 protein.

a. Adenoviral Infection

One method for delivery of the expression constructs for elevated expression of ZNF145 involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a transgenic construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them suitable mRNA's for translation.

Recombinant adenovirus may be generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. An example of a helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful transformation and expression of expression constructs for elevated expression of ZNF145. The adenovirus may be of any of the 42 different known serotypes or subgroups A-F. Adenovirus type 5 of subgroup C may be used as the starting material in order to obtain the conditional replication-defective adenovirus vector for use in the methods and compositions described here. This is because Adenovirus type 5 is a human adenovirus about which a great deal of biochemical and genetic information is known, and it has historically been used for most constructions employing adenovirus as a vector.

As stated above, the typical vector is replication defective and will not have an adenovirus E1 region. Thus, it will be most convenient to introduce the transforming construct for elevated expression of ZNF145 at the position from which the E1-coding sequences have been removed. However, the position of insertion of the expression construct for elevated expression of ZNF145 within the adenovirus sequences is not critical. The polynucleotide encoding ZNF145 may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10.sup.9-10.sup.11 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. AAV Infection

Adeno-associated virus (AAV) is an attractive vector system for use in the methods and compositions described here as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is suitable for use. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt, et al., 1994; Lebkowski, et al., 1988; Samulski, et al., 1989; Shelling and Smith, 1994; Yoder, et al., 1994; Zhou, et al., 1994; Hermonat and Muzyczka, 1984; Tratschin, et al., 1985; McLaughlin, et al., 1988) and genes involved in human diseases (Flotte, et al., 1992; Luo, et al., 1994; Ohi, et al., 1990; Walsh, et al., 1994; Wei, et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski, et al., 1989; McLaughlin, et al., 1988; Kotin, et al., 1990; Muzyczka, 1992).

Typically, recombinant AAV (rAAV) virus is made by cotransfecting a plasmid containing the gene of interest such as ZNF145 flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

c. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a transgene of interest such as ZNF145 is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

d. Lentivirus

Lentivirus vectors based on human immunodeficiency virus (HIV) type 1 (HIV-1) constitute a recent development in the field of gene therapy. A key property of HIV-1-derived vectors is their ability to infect nondividing cells. High-titer HIV-1-derived vectors have been produced. Examples of lentiviral vectors include White et al. (1999), describing a lentivirus vector which is based on HIV, simian immunodeficiency virus (SIV), and vesicular stomatitis virus (VSV) and which we refer to as HIV/SIVpack/G. The potential for pathogenicity with this vector system is minimal. The transduction ability of HIV/SIVpack/G was demonstrated with immortalized human lymphocytes, human primary macrophages, human bone marrow-derived CD34(+) cells, and primary mouse neurons. Gasmi et al. (1999) describe a system to transiently produce HIV-1-based vectors by using expression plasmids encoding gag, pol, and tat of HIV-1 under the control of the cytomegalovirus immediate-early promoter.

e. Other Viral Vectors

Other viral vectors may be employed as constructs. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Nucleic acids to be delivered, such as ZNF145 expression constructs, may also be housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

f. Non-Viral Transfer

DNA constructs such as expression vectors described here are generally delivered to a cell, in certain situations, the nucleic acid to be transferred is non-infectious, and can be transferred using non-viral methods.

Several non-viral methods for the transfer of expression constructs into cultured mammalian cells may be used. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nucleic acid encoding ZNF145 may be positioned and expressed at different sites. The nucleic acid ZNF145 may be stably integrated into the genome of the cell. This integration may be in the cognate location and orientation via homologous recombination (gene replacement) or it may be integrated in a random, non-specific location (gene augmentation). The nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

As an example, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

The liposome may be complexed with a hemagglutinating virus (HVJ). This has been shown to facilitate fusion with the cell membrane and promote cell entry of liposome-encapsulated DNA (Kaneda et al., 1989). The liposome may be complexed or employed in conjunction with nuclear nonhistone chromosomal proteins (HMG-1) (Kato et al., 1991). The liposome may be complexed or employed in conjunction with both HVJ and HMG-1. Such expression constructs have been successfully employed in transfer and expression of nucleic acid in vitro and in vivo.

Other vector delivery systems which can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

The delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, a nucleic acid encoding a therapeutic gene also may be specifically delivered into a cell type such as a mesenchymal stem cell, by any number of receptor-ligand systems with or without liposomes.

The expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO.sub.4 precipitated plasmids results in expression of the transfected genes. It is envisioned that DNA encoding a CAM may also be transferred in a similar manner in vivo and express CAM.

Another embodiment for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

### Expression of ZNF145

In mammalian host cells, a number of viral-based expression systems may be utilized. In cases where an adenovirus is used as an expression vector, sequences encoding ZNF145 may be ligated into an adenovirus transcription/translation complex consisting of the late promoter and tripartite leader sequence. Insertion in a non-essential E1 or E3 region of the viral genome may be used to obtain a viable virus which is capable of expressing ZNF145 in infected host cells. (Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells.

Thus, for example, the ZNF145 proteins may be expressed in either human embryonic kidney 293 (HEK293) cells or adherent dhfr CHO cells. To maximize receptor expression, typically all 5′ and 3′ untranslated regions (UTRs) are removed from the receptor cDNA prior to insertion into a pCDN or pCDNA3 vector. The cells are transfected with individual receptor cDNAs by lipofectin and selected in the presence of 400 mg/ml G418. After 3 weeks of selection, individual clones are picked and expanded for further analysis. HEK293 or CHO cells transfected with the vector alone serve as negative controls. To isolate cell lines stably expressing the individual receptors, about 24 clones are typically selected and analyzed by Northern blot analysis. Receptor mRNAs are generally detectable in about 50% of the G418-resistant clones analyzed.

As another example, ZNF145 may be introduced into a cell such as a mesenchymal stem cell by means of a lentiviral vector system. Thus, a ZNF145 sequence may be obtained for example from a suitable source, such as from cDNA of MSCs under osteogenesis, and cloned into a suitable vector such as pEntry3C (Invitrogen). Lentiviral vectors for over-expressing ZNF145 (i.e., lentiviral ZNF145 expression vectors) may be produced via recombination, for example, by LR recombination between this vector and pLenti6/V5 (Invitrogen). Lentivirus may be generated by co-transfecting the lentiviral ZNF145 expression vector with suitable packaging mix (e.g., from Invitrogen) into a suitable host cell, e.g., 293FT cells. Viral supernatant comprising lentiviral expression vectors is used to infect a chosen host cell, such as a mesenchymal stem cell. Infected cells are selected with, for example, 5 μg/ml blasticidin for 7 days.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding ZNF145. Such signals include the ATG initiation codon and adjacent sequences. In cases where sequences encoding ZNF145 and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used, such as those described in the literature. (Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and WI38), are available from the American Type Culture Collection (ATCC, Bethesda, Md.) and may be chosen to ensure the correct modification and processing of the foreign protein.

For long term, high yield production of recombinant proteins, stable expression may be employed. For example, cell lines capable of stably expressing ZNF145 can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes (Wigler, M. et al. (1977) Cell 11:223-32) and adenine phosphoribosyltransferase genes (Lowy, I. et al. (1980) Cell 22:817-23), which can be employed in tk⁻ or apr⁻ cells, respectively. Also, antimetabolite, antibiotic, or herbicide resistance can be used as the basis for selection. For example, dhfr confers resistance to methotrexate (Wigler, M. et al. (1980) Proc. Natl. Acad. Sci. 77:3567-70); npt confers resistance to the aminoglycosides neomycin and G-418 (Colbere-Garapin, F. et al (1981) J. Mol. Biol. 150:1-14); and als or pat confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine. (Hartman, S. C. and R. C. Mulligan (1988) Proc. Natl. Acad. Sci. 85:8047-51.) Recently, the use of visible markers has gained popularity with such markers as anthocyanins, β-glucuronidase and its substrate GUS, and luciferase and its substrate luciferin. These markers can be used not only to identify transformants, but also to quantify the amount of transient or stable protein expression attributable to a specific vector system. (Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding ZNF145 is inserted within a marker gene sequence, transformed cells containing sequences encoding ZNF145 can be identified by the absence of marker gene function. Alternatively, a marker gene can be placed in tandem with a sequence encoding ZNF145 under the control of a single promoter. Expression of the marker gene in response to induction or selection usually indicates expression of the tandem gene as well.

Alternatively, host cells which contain the nucleic acid sequence encoding ZNF145 and express ZNF145 may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

The presence of polynucleotide sequences encoding ZNF145 can be detected by DNA-DNA or DNA-RNA hybridization or amplification using probes or fragments or fragments of polynucleotides encoding ZNF145. Nucleic acid amplification based assays involve the use of oligonucleotides or oligomers based on the sequences encoding ZNF145 to detect transformants containing DNA or RNA encoding ZNF145.

A variety of protocols for detecting and measuring the expression of ZNF145, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on ZNF145 may be used, but a competitive binding assay may also be employed. These and other assays are well described in the art, for example, in Hampton, R. et al. (1990; Serological Methods, a Laboratory Manual, Section IV, APS Press, St Paul, Minn.) and in Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding ZNF145 include oligolabeling, nick translation, end-labeling, or PCR amplification using a labeled nucleotide. Alternatively, the sequences encoding ZNF145, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding ZNF145 may be cultured under conditions suitable for the expression and recovery of the protein from cell culture. The protein produced by a transformed cell may be located in the cell membrane, secreted or contained intracellularly depending on the sequence and/or the vector used. As will be understood by those of skill in the art, expression vectors containing polynucleotides which encode ZNF145 may be designed to contain signal sequences which direct secretion of ZNF145 through a prokaryotic or eukaryotic cell membrane. Other constructions may be used to join sequences encoding ZNF145 to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the ZNF145 encoding sequence may be used to facilitate purification. One such expression vector provides for expression of a fusion protein containing ZNF145 and a nucleic acid encoding 6 histidine residues preceding a thioredoxin or an enterokinase cleavage site. The histidine residues facilitate purification on immobilized metal ion affinity chromatography (IMIAC; described in Porath, J. et al. (1992) Prot. Exp. Purif. 3: 263-281), while the enterokinase cleavage site provides a means for purifying ZNF145 from the fusion protein. A discussion of vectors which contain fusion proteins is provided in Kroll, D. J. et al. (1993; DNA Cell Biol. 12:441-453).

Fragments of ZNF145, as well as whole length polypeptides, may be produced not only by recombinant production, but also by direct peptide synthesis using solid-phase techniques. (Merrifield J. (1963) J. Am. Chem. Soc. 85:2149-2154.) Protein synthesis may be performed by manual techniques or by automation. Automated synthesis may be achieved, for example, using the Applied Biosystems 431A peptide synthesizer (Perkin Elmer). Various fragments of ZNF145 may be synthesized separately and then combined to produce the full length molecule.

Other methods of expression are also known, for example, a method known as “gene activation” may be employed to modulate activity or expression of ZNF145. This method is described in detail in U.S. Pat. No. 5,641,670, hereby incorporated by reference. In essence, the gene activation method is based upon the recognition that the regulation or activity of endogenous genes of interest in a cell can be altered by inserting into the cell genome, at a preselected site, through homologous recombination, a suitable DNA construct comprising: (a) a targeting sequence; (b) a regulatory sequence; (c) an exon and (d) an unpaired splice-donor site, wherein the targeting sequence directs the integration of elements (a)-(d) such that the elements (b)-(d) are operatively linked to the endogenous gene. The DNA construct may alternatively comprise: (a) a targeting sequence, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the first exon of the endogenous gene.

The targeting sequences used are selected with reference to the site into which the DNA is to be inserted. In both arrangements the targeting event is used to create a new transcription unit, which is a fusion product of sequences introduced by the targeting DNA constructs and the endogenous cellular gene. For example, the formation of the new transcription unit allows transcriptionally silent genes (genes not expressed in a cell prior to transfection) to be activated in host cells by introducing into the host cell's genome a DNA construct as described. The expression of an endogenous gene such as ZNF145 which is expressed in a cell as obtained can be altered in that it is increased, reduced, including eliminated, or the pattern of regulation or induction may be changed through use of the gene activation method.

### ZNF145 Agonists

Identifying ZNF145 Modulators, Agonists and Antagonists

Agonists, in particular, small molecules may be used to specifically enhance the activity or expression of ZNF145 for use as chondrogenesis-promoting agents.

We therefore disclose ZNF145 agonist, which may be small molecules, as well as assays for screening for these. Agonists of ZNF145 may be screened by detecting modulation, such as up regulation, of binding or other ZNF145 activity. We therefore provide a compound capable of up-regulating the expression, amount or activity of a ZNF145 polypeptide. Such a compound may be used in the methods and compositions described here for promoting chondrogenesis, cartilage, bone or ligament repair, regeneration, treatment of a degenerative disease, etc.

ZNF145 may therefore be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2): Chapter 5 (1991). Furthermore, screens may be conducted to identify factors which influence the expression of ZNF145, in particular in chondrogenic progenitor stem cells such as mesenchymal stem cells.

In general, the assays for agonists rely on determining the effect of candidate molecules on one or more activities of ZNF145. An assay may involve assaying ZNF145 activity in the presence of a candidate molecule, and optionally in the absence of the candidate molecule, or in the presence of a molecule known to inhibit or activate a ZNF145 activity.

We have demonstrated that expression of ZNF145 is increased in chondrogenic mesenchymal stem cells; accordingly, control of ZNF145 expression may be employed to promote chondrogenesis. Therefore, it is desirous to find compounds and drugs which stimulate the expression and/or activity of ZNF145, or which can inhibit the function of this protein. In general, agonists and antagonists are employed for therapeutic and prophylactic purposes for any known degenerative disease.

By “up-regulation” we include any positive effect on the behaviour being studied; this may be total or partial. Thus, where binding is being detected, candidate agonists are capable of enhancing, promoting, or making stronger the binding between two entities. The up-regulation of binding (or any other activity) achieved by the candidate molecule may be at least 10%, such as at least 20%, at least 30%, at least 40%, at least 50%, at least 60%, at least 70%, at least 80%, at least 90% or more compared to binding (or which ever activity) in the absence of the candidate molecule. Thus, a candidate molecule suitable for use as an agonist is one which is capable of increasing by 10% more the binding or other activity.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. The compound may be an antibody.

Examples of potential antagonists of ZNF145 include small molecules, nucleotides and their analogues, including purines and purine analogues, oligonucleotides or proteins which are closely related to a binding partner of ZNF145, e.g., a fragment of the binding partner, or small molecules which bind to the ZNF145 but do not elicit a response, so that the activity of the polypeptide is prevented, etc.

Screening Kits

The materials necessary for such screening to be conducted may be packaged into a screening kit.

Such a screening kit is useful for identifying agonists, antagonists, ligands, receptors, substrates, enzymes, etc. for ZNF145 polypeptides or compounds which decrease or enhance the production of ZNF145. The screening kit may comprise: (a) a ZNF145 polypeptide; (b) a recombinant cell expressing a ZNF145 polypeptide; or (c) an antibody to ZNF145 polypeptide.

Antibodies against ZNF145 are known in the art and are commercially available, for example Rabbit anti-Human PML Polyclonal Antibody-a (Catalogue No AI70002A) and Rabbit anti-Human PML Polyclonal Antibody-b (AI70002B) from Anogen, Mississauga, Ontario, Canada).

The screening kit may comprise a library. The screening kit may comprise any one or more of the components needed for screening, as described below. The screening kit may optionally comprise instructions for use.

Screening kits may also be provided which are capable of detecting ZNF145 expression at the nucleic acid level. Such kits may comprise a primer for amplification of ZNF145, or a pair of primers for amplification. The primer or primers may be chosen from any suitable sequence, for example a portion of the ZNF145 sequence. Methods of identifying primer sequences are well known in the art, and the skilled person will be able to design such primers with ease. The kits may comprise a nucleic acid probe for ZNF145 expression, as described in this document. The kits may also optionally comprise instructions for use.

Rational Design

Rational design of candidate compounds likely to be able to interact with ZNF145 may be based upon structural studies of the molecular shapes of a ZNF145 polypeptide. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

Polypeptide Binding Assays

Modulators and antagonists of ZNF145 activity or expression may be identified by any means known in the art.

In their simplest form, the assays may simply comprise the steps of mixing a candidate compound with a solution containing a ZNF145 polypeptide to form a mixture, measuring activity of ZNF145 polypeptide in the mixture, and comparing the activity of the mixture to a standard.

Furthermore, molecules may be identified by their binding to ZNF145, in an assay which detects binding between ZNF145 and the putative molecule.

One type of assay for identifying substances that bind to a ZNF145 polypeptide described here involves contacting the ZNF145 polypeptide, which is immobilised on a solid support, with a non-immobilised candidate substance determining whether and/or to what extent the ZNF145 polypeptide of interest and candidate substance bind to each other. Alternatively, the candidate substance may be immobilised and the ZNF145 polypeptide as set out in this document non-immobilised.

The binding of the substance to the ZNF145 polypeptide can be transient, reversible or permanent. The substance may bind to the polypeptide with a Kd value which is lower than the Kd value for binding to control polypeptides (e.g., polypeptides known to not be involved in chondrogenesis). The Kd value of the substance may be 2 fold less than the Kd value for binding to control polypeptides, such as a Kd value 100 fold less or a Kd 1000 fold less than that for binding to the control polypeptide.

In an example assay method, the ZNF145 polypeptide may be immobilised on beads such as agarose beads. Typically this may be achieved by expressing the ZNF145 polypeptide as a GST-fusion protein in bacteria, yeast or higher eukaryotic cell lines and purifying the GST-ZNF145 fusion protein from crude cell extracts using glutathione-agarose beads (Smith and Johnson, 1988; Gene 67(10):31-40). As a control, binding of the candidate substance, which is not a GST-fusion protein, to an immobilised polypeptide may be determined in the absence of the ZNF145 polypeptide. The binding of the candidate substance to the immobilised ZNF145 polypeptide may then be determined. This type of assay is known in the art as a GST pulldown assay. Again, the candidate substance may be immobilised and the ZNF145 polypeptide non-immobilised.

It is also possible to perform this type of assay using different affinity purification systems for immobilising one of the components, for example Ni-NTA agarose and histidine-tagged components.

Binding of the polypeptide to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, such as from 1 to 100 nmol/ml. In the case of antibodies, the final concentration used is typically from 100 to 500 μg/ml, such as from 200 to 300 μg/ml.

Modulators and antagonists of ZNF145 may also be identified by detecting modulation of binding between ZNF145 and any molecule to which this polypeptide binds, or modulation of any activity consequential on such binding or release.

Cell Based Assays

A cell based assay may simply test binding of a candidate compound wherein adherence to the cells bearing the ZNF145 polypeptide is detected by means of a label directly or indirectly associated with the candidate compound or in an assay involving competition with a labeled competitor.

Further, these assays may test whether the candidate compound results in a signal generated by binding to the ZNF145 polypeptide, using detection systems appropriate to the cells bearing the polypeptides at their surfaces. Inhibitors of activation are generally assayed in the presence of a known agonist and the effect on activation by the agonist by the presence of the candidate compound is observed.

Another method of screening compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses.

Competitive assays are particularly useful, where the cells expressing the library of compounds are contacted or incubated with a labelled antibody known to bind to a ZNF145 polypeptide, such as ¹²⁵I-antibody, and a test sample such as a candidate compound whose binding affinity to the binding composition is being measured. The bound and free labelled binding partners for the ZNF145 polypeptide are then separated to assess the degree of binding. The amount of test sample bound is inversely proportional to the amount of labelled antibody binding to the ZNF145 polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

The assays may involve exposing a candidate molecule to a cell, such as a chondrogenic progenitor stem cell, for example, a mesenchymal stem cell, and assaying expression of ZNF145 by any suitable means. Molecules which up-regulate the expression of ZNF145 in such assays may be optionally chosen for further study, and used as drugs to up-regulate ZNF145 expression. Such drugs may be usefully employed to treat or prevent degenerative disease, or for promoting repair or regneration of cartilage, bone or ligament, etc.

cDNA encoding ZNF145 protein and antibodies to the proteins may also be used to configure assays for detecting the effect of added compounds on the production of ZNF145 mRNA and protein in cells. For example, an ELISA may be constructed for measuring secreted or cell associated levels of ZNF145 polypeptide using monoclonal and polyclonal antibodies by standard methods known in the art, and this can be used to discover agents which may inhibit or enhance the production of ZNF145 protein (also called antagonist or agonist, respectively) from suitably manipulated cells or tissues. Standard methods for conducting screening assays are well understood in the art.

Activity Assays

Assays to detect modulators or antagonists typically involve detecting modulation of any activity of ZNF145, in the presence, optionally together with detection of modulation of activity in the absence, of a candidate molecule.

The activity that may be detected can comprise any ZNF145-dependent activity, such as binding activity. ZNF145 is known to bind to UDP-N-acetylglucosamine 2-epimerase/N-acetylmannosamine kinase (GNE) (Weidemann et al, FEBS Lett. 2006 Dec. 11; 580(28-29):6649-54), and binding activity of ZNF145 to UDP-N-acetylglucosamine 2-epimerase/N-acetylmannosamine kinase (GNE) may be assayed by means known in the art, for example, GST-pulldown assays. One of ZNF145 and UDP-N-acetylglucosamine 2-epimerase/N-acetylmannosamine kinase (GNE) may be immobilised and the other radiolabelled. Binding of ZNF145 to UDP-N-acetylglucosamine 2-epimerase/N-acetylmannosamine kinase (GNE) may then be detected by assaying captured radioactivity on exposure of ZNF145 to UDP-N-acetylglucosamine 2-epimerase/N-acetylmannosamine kinase (GNE).

Assays which detect specific biological activities of ZNF145 may also be used. The assays typically involve contacting a candidate molecule (e.g., in the form of a library) with ZNF145 whether in the form of a polypeptide, a nucleic acid encoding the polypeptide, or a cell, organelle, extract, or other material comprising such, with a candidate modulator. The relevant activity of ZNF145 (as described below) may be detected, to establish whether the presence of the candidate modulator has any effect.

Known activities of ZNF145, any one or more of which may be used for assaying ZNF134 activity, include DNA binding, metal ion binding, protein homodimerization activity, specific transcriptional repressor activity and zinc ion binding. Assays for each of these activities are well known in the art. Processes in which ZNF145 is involved include apoptosis, central nervous system development, mesonephros development, negative regulation of myeloid cell differentiation, negative regulation of transcription, DNA-dependent, transcription and ubiquitin cycle. Methods of assaying these processes are also known in the art.

The assays described above may be performed in the presence or absence of a candidate modulator and the appropriate activity detected to detect modulation of ZNF145 activity and hence identification of a candidate modulator and/or antagonist of ZNF145.

Promoter binding assays to detect candidate modulators which bind to and/or affect the transcription or expression of ZNF145 may also be used. Candidate modulators may then be chosen for further study, or isolated for use. Details of such screening procedures are well known in the art, and are for example described in, Handbook of Drug Screening, edited by Ramakrishna Seethala, Prabhavathi B. Fernandes (2001, New York, N.Y., Marcel Dekker, ISBN 0-8247-0562-9).

The screening methods described here may employ in vivo assays, although they may be configured for in vitro use. In vivo assays generally involve exposing a cell comprising ZNF145 to the candidate molecule. In in vitro assays, ZNF145 is exposed to the candidate molecule, optionally in the presence of other components, such as crude or semi-purified cell extract, or purified proteins. Where in vitro assays are conducted, these may employ arrays of candidate molecules (for example, an arrayed library). In vivo assays may be employed. Therefore, ZNF145 polypeptide may be comprised in a cell, such as heterologously. Such a cell may be a transgenic cell, which has been engineered to express ZNF145 as described above.

Where an extract is employed, it may comprise a cytoplasmic extract or a nuclear extract, methods of preparation of which are well known in the art.

It will be appreciated that any component of a cell comprising ZNF145 may be employed, such as an organelle. One embodiment utilises a cytoplasmic or nuclear preparation, e.g., comprising a cell nucleus which comprises ZNF145 as described. The nuclear preparation may comprise one or more nuclei, which may be permeabilised or semi-permeabilised, by detergent treatment, for example.

Thus, in a specific embodiment, an assay format may include the following: a multiwell microtitre plate is set up to include one or more cells expressing ZNF145 polypeptide in each well; individual candidate molecules, or pools of candidate molecules, derived for example from a library, may be added to individual wells and modulation of ZNF145 activity measured. Where pools are used, these may be subdivided in to further pools and tested in the same manner. ZNF145 activity, for example binding activity or transcriptional co-activation activity, as described elsewhere in this document may then be assayed.

Alternatively or in addition to the assay methods described above, “subtractive” procedures may also be used to identify modulators or antagonists of ZNF145. Under such “subtractive” procedures, a plurality of molecules is provided, which comprises one or more candidate molecules capable of functioning as a modulator (e.g., cell extract, nuclear extract, library of molecules, etc), and one or more components is removed, depleted or subtracted from the plurality of molecules. The “subtracted” extract, etc, is then assayed for activity, by exposure to a cell comprising ZNF145 (or a component thereof) as described.

Thus, for example, an ‘immunodepletion’ assay may be conducted to identify such modulators as follows. A cytoplasmic or nuclear extract may be prepared from a pluripotent cell, for example, a pluripotent EG/ES cell. The extract may be depleted or fractionated to remove putative modulators, such as by use of immunodepletion with appropriate antibodies. If the extract is depleted of a modulator, it will lose the ability to affect ZNF145 function or activity or expression. A series of subtractions and/or depletions may be required to identify the modulators or antagonists.

It will also be appreciated that the above “depletion” or “subtraction” assay may be used as a preliminary step to identify putative modulatory factors for further screening. Furthermore, or alternatively, the “depletion” or “subtraction” assay may be used to confirm the modulatory activity of a molecule identified by other means (for example, a “positive” screen as described elsewhere in this document) as a putative modulator.

Candidate molecules subjected to the assay and which are found to be of interest may be isolated and further studied. Methods of isolation of molecules of interest will depend on the type of molecule employed, whether it is in the form of a library, how many candidate molecules are being tested at any one time, whether a batch procedure is being followed, etc.

The candidate molecules may be provided in the form of a library. In one embodiment, more than one candidate molecule may be screened simultaneously. A library of candidate molecules may be generated, for example, a small molecule library, a polypeptide library, a nucleic acid library, a library of compounds (such as a combinatorial library), a library of antisense molecules such as antisense DNA or antisense RNA, an antibody library etc, by means known in the art. Such libraries are suitable for high-throughput screening. Different cells comprising ZNF145 may be exposed to individual members of the library, and effect on the ZNF145 activity determined. Array technology may be employed for this purpose. The cells may be spatially separated, for example, in wells of a microtitre plate.

In an embodiment, a small molecule library is employed. By a “small molecule”, we refer to a molecule whose molecular weight may be less than about 50 kDa. In particular embodiments, a small molecule may have a molecular weight which is less than about 30 kDa, such as less than about 15 kDa or less than 10 kDa or so. Libraries of such small molecules, here referred to as “small molecule libraries” may contain polypeptides, small peptides, for example, peptides of 20 amino acids or fewer, for example, 15, 10 or 5 amino acids, simple compounds, etc.

Alternatively or in addition, a combinatorial library, as described in further detail below, may be screened for modulators or antagonists of ZNF145. Assays for ZNF145 activity are described above.

Libraries

Libraries of candidate molecules, such as libraries of polypeptides or nucleic acids, may be employed in the screens for ZNF145 antagonists and inhibitors described here. Such libraries are exposed to ZNF145 protein, and their effect, if any, on the activity of the protein determined.

Selection protocols for isolating desired members of large libraries are known in the art, as typified by phage display techniques. Such systems, in which diverse peptide sequences are displayed on the surface of filamentous bacteriophage (Scott and Smith (1990 supra), have proven useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference). Such techniques may be modified if necessary for the expression generally of polypeptide libraries.

One particularly advantageous approach has been the use of scFv phage-libraries (Bird, R. E., et al. (1988) Science 242: 423-6, Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) supra; Marks et al. (1991) supra; Chiswell et al. (1992) Trends Biotech., 10: 80; Marks et al. (1992) supra). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra), which are incorporated herein by reference.

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the methods and compositions described here. These expression systems may be used to screen a large number of different members of a library, in the order of about 10⁶ or even more. Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, W090/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

The library may in particular comprise a library of zinc fingers; zinc fingers are known in the art and act as transcription factors. Suitable zinc finger libraries are disclosed in, for example, WO 96/06166 and WO 98/53057. Construction of zinc finger libraries may utilise rules for determining interaction with specific DNA sequences, as disclosed in for example WO 98/53058 and WO 98/53060. Zinc fingers capable of interacting specifically with methylated DNA are disclosed in WO 99/47656. The above zinc finger libraries may be immobilised in the form of an array, for example as disclosed in WO 01/25417.

Candidate molecules subjected to the assay and which are found to be of interest may be isolated and further studied. Methods of isolation of molecules of interest will depend on the type of molecule employed, whether it is in the form of a library, how many candidate molecules are being tested at any one time, whether a batch procedure is being followed, etc.

The candidate molecules may be provided in the form of a library. In an embodiment, more than one candidate molecule is screened simultaneously. A library of candidate molecules may be generated, for example, a small molecule library, a polypeptide library, a nucleic acid library, a library of compounds (such as a combinatorial library), a library of antisense molecules such as antisense DNA or antisense RNA, an antibody library etc, by means known in the art. Such libraries are suitable for high-throughput screening. Chondrogenic progenitor cells such as mesenchymal stem cells may be exposed to individual members of the library, and the effect on chondrogenesis, if any, cell determined. Array technology may be employed for this purpose. The cells may be spatially separated, for example, in wells of a microtitre plate.

In an embodiment, a small molecule library is employed. By a “small molecule”, we refer to a molecule whose molecular weight may be less than about 50 kDa. In particular embodiments, a small molecule has a molecular weight may be less than about 30 kDa, such as less than about 15 kDa, or less than 10 kDa or so. Libraries of such small molecules, here referred to as “small molecule libraries” may contain polypeptides, small peptides, for example, peptides of 20 amino acids or fewer, for example, 15, 10 or 5 amino acids, simple compounds, etc.

### Detection of Chrondrogenic Mesenchymal Stem Cells in Cell Populations

Polynucleotide probes or antibodies as described here may be used for the detection of mesenchymal stem cells which are chondrogenic, have chondrogenic potential, or are capable of differentiating in a chondrogenic pathway in cell populations. As used herein, a “cell population” is any collection of cells which may contain one or more cells such as mesenchymal stem cells. For example, the collection of cells may not consist solely of mesenchymal stem cells, but may comprise at least one other cell type.

Cell populations comprise embryos and embryo tissue, but also adult tissues and tissues grown in culture and cell preparations derived from any of the foregoing.

Polynucleotides as described here may be used for detection of ZNF145 transcripts in mesenchymal stem cells by nucleic acid hybridisation techniques. Such techniques include PCR, in which primers are hybridised to ZNF145 transcripts and used to amplify the transcripts, to provide a detectable signal; and hybridisation of labelled probes, in which probes specific for an unique sequence in the ZNF145 transcript are used to detect the transcript in the target cells.

As noted hereinbefore, probes may be labelled with radioactive, radioopaque, fluorescent or other labels, as is known in the art.

Antibodies against ZNF145, which may be generated by means known in the art, may also be used to detect ZNF145. For example, intracellular scFv may be used to detect ZNF145 within the cell.

Particularly indicated are immunostaining and FACS techniques. Suitable fluorophores are known in the art, and include chemical fluorophores and fluorescent polypeptides, such as GFP and mutants thereof (see WO 97/28261). Chemical fluorophores may be attached to immunoglobulin molecules by incorporating binding sites therefor into the immunoglobulin molecule during the synthesis thereof.

The fluorophore may comprise a fluorescent protein, which is advantageously GFP or a mutant thereof. GFP and its mutants may be synthesised together with the immunoglobulin or target molecule by expression therewith as a fusion polypeptide, according to methods well known in the art. For example, a transcription unit may be constructed as an in-frame fusion of the desired GFP and the immunoglobulin or target, and inserted into a vector as described above, using conventional PCR cloning and ligation techniques.

Antibodies against ZNF145 may be labelled with any label capable of generating a signal. The signal may be any detectable signal, such as the induction of the expression of a detectable gene product. Examples of detectable gene products include bioluminescent polypeptides, such as luciferase and GFP, polypeptides detectable by specific assays, such as β-galactosidase and CAT, and polypeptides which modulate the growth characteristics of the host cell, such as enzymes required for metabolism such as HIS3, or antibiotic resistance genes such as G418. For example, he signal may be detectable at the cell surface or within the cell. For example, the signal may be a luminescent or fluorescent signal, which is detectable from outside the cell and allows cell sorting by FACS or other optical sorting techniques.

Optical immunosensor technology, based on optical detection of fluorescently-labelled antibodies, may be employed. Immunosensors are biochemical detectors comprising an antigen or antibody species coupled to a signal transducer which detects the binding of the complementary species (Rabbany et al., 1994 Crit Rev Biomed Eng 22:307-346; Morgan et al., 1996 Clin Chem 42:193-209). Examples of such complementary species include the antigen Zif268 and the anti-Zif268 antibody. Immunosensors produce a quantitative measure of the amount of antibody, antigen or hapten present in a complex sample such as serum or whole blood (Robinson 1991 Biosens Bioelectron 6:183-191). The sensitivity of immunosensors makes them ideal for situations requiring speed and accuracy (Rabbany et al., 1994 Crit Rev Biomed Eng 22:307-346).

Detection techniques employed by immunosensors include electrochemical, piezoelectric or optical detection of the immuno interaction (Ghindilis et al., 1998 Biosens Bioelectron 1:113-131). An indirect immunosensor uses a separate labelled species that is detected after binding by, for example, fluorescence or luminescence (Morgan et al., 1996 Clin Chem 42:193-209). Direct immunosensors detect the binding by a change in potential difference, current, resistance, mass, heat or optical properties (Morgan et al., 1996 Clin Chem 42:193-209). Indirect immunosensors may encounter fewer problems due to non-specific binding (Attridge et al., 1991 Biosens Bioelecton 6:201-214; Morgan et al., 1996 Clin Chem 42:193-209).

### Pharmaceutical Compositions

The chondrogenesis-promoting agents described here, including ZNF145 nucleic acids, ZNF145 polypeptides, ZNF145 agonists and ZNF145 over-expressing cells, may be produced in large amounts at low cost in a bioactive form, allowing the development of chondrogenesis-promoting agent containing formulations by aerosolisation, nebulisation, intranasal or intratracheal administration, etc.

While it is possible for the composition comprising the chondrogenesis-promoting agent to be administered alone, the active ingredient may be formulated as a pharmaceutical formulation. We therefore also disclose pharmaceutical compositions comprising a chondrogenesis-promoting agent described here, including one or more of ZNF145 nucleic acids, ZNF145 polypeptides, ZNF145 agonists and ZNF145 over-expressing cells. Such pharmaceutical compositions are useful for delivery of chondrogenesis-promoting agents to an individual for the treatment or alleviation of symptoms as described.

The composition may include the chondrogenesis-promoting agent, including a ZNF145 nucleic acid, a ZNF145 polypeptide, a ZNF145 agonist and ZNF145 over-expressing cell, a structurally related compound, or an acidic salt thereof The pharmaceutical formulations comprise an effective amount of chondrogenesis-promoting agent, such as a ZNF145 nucleic acid, a ZNF145 polypeptide, a ZNF145 agonist and ZNF145 over-expressing cell, together with one or more pharmaceutically-acceptable carriers. An “effective amount” of an chondrogenesis-promoting agent, such as a ZNF145 nucleic acid, a ZNF145 polypeptide, a ZNF145 agonist and ZNF145 over-expressing cell thereof is the amount sufficient to alleviate at least one symptom of a disease as described, such as atopic allergy.

The effective amount will vary depending upon the particular disease or syndrome to be treated or alleviated, as well as other factors including the age and weight of the patient, how advanced the disease etc state is, the general health of the patient, the severity of the symptoms, and whether the chondrogenesis-promoting agent, such as a ZNF145 nucleic acid, a ZNF145 polypeptide, a ZNF145 agonist and ZNF145 over-expressing cell is being administered alone or in combination with other therapies.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions disclosed here include those suitable for topical and oral administration, with topical formulations being for example used where the tissue affected is primarily the skin or epidermis (for example, psoriasis, eczema and other epidermal diseases). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the chondrogenesis-promoting agent, such as a ZNF145 nucleic acid, a ZNF145 polypeptide, a ZNF145 agonist and ZNF145 over-expressing cell in the topical formulation is in an amount of about 0.5 to 50% by weight of the composition, such as about 1 to 30%, about 2-20% or about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of active ingredient must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the agent can be incorporated into the formulation to increase the penetration of the active compound(s) from the skin surface into epidermal keratinocytes. A skin enhancer which increases the absorption of the active compound(s) into the skin reduces the amount of agent needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone.®. (Hadgraft, Eur. J. Drug. Metab. Pharmacokinet, 21:165-173 (1996)); alcohols, sulphoxides, fatty acids, esters, Azone.®., pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone.®. and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of an agent or composition can be determined by techniques known to those of skill in the art. For example, radiolabeling of the active compound, followed by measurement of the amount of radiolabeled compound absorbed by the skin enables one of skill in the art to determine levels of the composition absorbed using any of several methods of determining skin penetration of the test compound. Publications relating to skin penetration studies include Reinfenrath, W G and G S Hawkins. The Weaning Yorkshire Pig as an Animal Model for Measuring Percutaneous Penetration. In:Swine in Biomedical Research (M. E. Tumbleson, Ed.) Plenum, N.Y., 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp. 67-80; and W. G. Reifenrath, Cosmetics & Toiletries, 110:3-9 (1995).

For some applications, a long acting form of agent or composition may be administered using formulations known in the art, such as polymers. The agent can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

The pharmaceutical composition can be administered in an oral formulation in the form of tablets, capsules or solutions. An effective amount of the oral formulation is administered to patients 1 to 3 times daily until the symptoms of the disease alleviated. The effective amount of agent depends on the age, weight and condition of a patient. In general, the daily oral dose of agent is less than 1200 mg, and more than 100 mg. The daily oral dose may be about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, suppositories. In general, the formulations are prepared by uniformly and intimately bringing into association the agent composition with liquid carriers or finely divided solid carriers or both, and as necessary, shaping the product. The active ingredient can be incorporated into a variety of basic materials in the form of a liquid, powder, tablets or capsules to give an effective amount of active ingredient to treat the disease.

Other therapeutic agents suitable for use herein are any compatible drugs that are effective for the intended purpose, or drugs that are complementary to the agent formulation. The formulation utilized in a combination therapy may be administered simultaneously, or sequentially with other treatment, such that a combined effect is achieved.

### Administration of ZNF-145 Over-Expressing Cells

We Describe the Administration of ZNF145 Over-Expressing Cells to an Individual

Therapeutic or prophylactic treatment of an individual with ZNF145 over-expressing cells may be considered efficacious if a disease, disorder or condition is measurably improved in any way. Such improvement may be shown by a number of indicators. Measurable indicators include, for example, detectable changes in a physiological condition or set of physiological conditions associated with a particular disease, disorder or condition (including, but not limited to, blood pressure, heart rate, respiratory rate, counts of various blood cell types, levels in the blood of certain proteins, carbohydrates, lipids or cytokines or modulation expression of genetic markers associated with the disease, disorder or condition). Treatment of an individual with the ZNF145 over-expressing cells would be considered effective if any one of such indicators responds to such treatment by changing to a value that is within, or closer to, the normal value. The normal value may be established by normal ranges that are known in the art for various indicators, or by comparison to such values in a control. In medical science, the efficacy of a treatment is also often characterized in terms of an individual's impressions and subjective feeling of the individual's state of health. Improvement therefore may also be characterized by subjective indicators, such as the individual's subjective feeling of improvement, increased well-being, increased state of health, improved level of energy, or the like, after administration of ZNF145 over-expressing cells as described here.

The ZNF145 over-expressing cells described here may be administered to a patient in any pharmaceutically or medically acceptable manner, including by injection or transfusion. The ZNF145 over-expressing cells may contain, or be contained in any pharmaceutically-acceptable carrier. The ZNF145 over-expressing cells may be carried, stored, or transported in any pharmaceutically or medically acceptable container, for example, a blood bag, transfer bag, plastic tube or vial.

### EXAMPLES

### Example 1

### MSC Culture and Osteoblast, Chondrocyte and Adipocyte Differentiation

Human bone marrow-derived mesenchymal stem cells (hBMSCs) are harvested from the iliac crest and cultured as described (Sekiya et al., 2002) after informed consent according to guidelines of the National University Hospital of Singapore.

To prevent spontaneous differentiation, cells are maintained at subconfluent levels. MSCs are induced to differentiate towards adipocytes and osteoblasts as described (Liu et al., 2007), 2×10⁵ and 1.5×10⁵ MSCs in W6 plate are induced to differentiate into adipocytes and osteoblasts for 14 days in adipogenic and osteogenic medium, respectively.

Adipogenic medium contained 0.5 mM isobutyl-methylxanthine (IBMX), 1 μM dexamethasone (Sigma), 10 μM insulin, 200 μM indomethacin, and 1% antibiotic/antimycotic. Osteogenic medium contained 0.1 μM dexamethasone, 50 μM ascorbate-2-phosphate, 10 mM β-glycerophosphate, and 1% antibiotic/antimycotic.

Pellet culture system described (Liu et al., 2007) is used for chondrocyte differentiation. Briefly, 2×10⁵ MSCs are placed in a 15 ml polypropylene tube (Falcon) and centrifuged to a pellet. The pellet is cultured at 37° C. with 5% CO₂ in 500 μl of chondrogenic medium that contained 10 ng/ml transforming growth factor (TGF)-β3, 10⁻⁷ M dexamethasone, 50 μg/ml ascorbate-2-phosphate, 40 μg/ml proline, 100 μg/ml pyruvate, and 50 mg/ml ITS+Premix (Becton Dickinson; 6.25 μg/ml insulin, 6.25 μg/ml transferrin, 6.25 βg/ml selenious acid, 1.25 mg/ml BSA, and 5.35 mg/ml linoleic acid). The medium is replaced every 3-4 days for 28 days.

Differentiation of MSCs is evaluated by real time PCR and stain. Oil red O stain for lipoid deposits in adipogenesis, alizarin red S stain for calcium deposition in osteogenesis, immunostaining against collagen type 2 (COL2A1) and alcian blue stain for cartilage proteoglycans in chondrogenesis is used in this study.

### Example 2

### Construction of Expression Plasmids and Infection into MSCs

ZNF145 is amplified from cDNA of MSCs under osteogenesis for 14 days and then cloned into pEntry3C (Invitrogen).

Sox9 ultimate ORF clone is from Invitrogen. Via LR recombination between pEntry3C and pLenti6/V5 (Invitrogen), pLentiviral vectors for overexpressing ZNF145 or Sox9 are created. Lentivirus is generated by cotransfecting pLentiviral vector for overexpressing ZNF145 or Sox9 and packaging mix (Invitrogen) into 293FT cells, then MSCs are infected with viral supernatant to achieve ZNF145 or Sox9 overexpression and are selected with 5 μg/ml blasticidin for 7 d. The empty pLenti6/V5 with no insert is used as control (Empty).

### Example 3

### Quantitative Real Time PCR

To quantify effect of ZNF145 overexpression or knockdown on differentiation of MSCs, quantitative real time PCR is performed with Taqman expression assay according to the manufacturer and an ABI 7700 Prism (Applied Biosystems).

Briefly, 0.3 μg of total RNA is converted to cDNA using high capacity cDNA archive kit in 30 ul and then diluted to 300 μl. Quantitative RT-PCR is done as follows: intial denaturation for 2 min at 50° C., 10 min at 95° C., following 40 cycles of PCR (95° C. for 15 s, 60° C. for 1 min) by using 5 μl of 2× Master mix, 0.5 μl of Taqman probe and 4.5 μl of cDNA.

All probes are designed with a 5′ fluoregenic 6-carboxylfluorescein, and a 3′ quencher, tetramethyl-6-carboxyrhodamine. The expression of human GAPDH is used to normalize gene expression levels.

### Example 4

### Indirect Immunofluoscenct Cell Staining

Cells growing on chambers are washed with PBS and fixed with 10% neutral formalin for 15 min at room temperature.

After two washes with Rinse buffer (1×TBS+0.05% Tween 20), cells are permeabilized with 0.1% Triton X-100/PBS for 10 min. The cells are treated with 4% goat serum (blocking buffer) for 30 min and then incubated for 1 h with primary antibody against ZNF145 diluted 1:50 in blocking buffer.

After 3 washes with Rinse buffer, the cells are incubated with FITC-conjugated secondary antibody diluted 1:150 in PBS for 45 min. After three washes, immunolocation is examined with a fluorescence microscope (Olympus, Tokyo, Japan).

### Example 5

### Western Blot Analysis

Cells are collected by centrifugation, cell pellet is resuspended in lysis buffer (25 mM Tris, pH7.5, 150 mM NaCl, 1% NP-40, 0.5% sodium deoxycholate, 0.1% SDS) containing proteinase inhibitors and incubated on ice for 30 min.

Following centrifugation at 16000 g for 15 min at 4° C., the supernatant containing total cell extract is collected and kept at −80° C. Protein from cell extracts in the gel is electrophoretically transferred onto a Hybond-PVDF membrane (Amersham Biosciences). The membrane is incubated for 1 h at room temperature in blocking buffer (TBS-T containing 5% skim milk) to block nonspecific protein binding and then incubated at room temperature for 1 h with the primary antibody against ZNF145 or Sox9 (Santa Cruz) diluted (1:300) in blocking buffer for 1 h.

Following four washes with TBS-T, the membrane is incubated for 1 h with the HPR-conjugated secondary antibody diluted (1:3000) in blocking buffer for 1 h. Antibody binding is visualized with an ECL Western blotting detection system (Amersham Biosciences).

### Example 6

### cDNA Microarray Analysis

To determine the targets of ZNF145 in MSCs, we overexpressed ZNF145 in MSCs and analyzed their gene expression profiles in undifferentiated MSCs using microarrays.

Total RNA is isolated from ZNF145-overexpressing and no insert control MSCs using RNeasy mini-kit (Qiagen, Chatsworth, Calif.) per the manufacturer's protocol. In brief, 3.5 μg total RNA is used to synthesize double-strand DNA using one cycle cDNA synthesis kit. cDNA is purified by using Sample Cleanup Module. In vitro transcription is perfomed to produce biotin-labeled cRNA using GeneChip IVT Labeling Kit. Biotinylated cRNA is cleaned and fragmented to 50-200 nucleotides with Sample Cleanup Module and hybridized 16 h at 45° C. to Affymetrix HG U133 plus 2 containing more than 54675 human genes.

After washing, the array is stained with streptavidin-phycoerythrin (Molecular Probes). The staining signal is amplified by biotinylated anti-streptavidin (Vector Laboratories), followed by streptavidin-phycoerythrin stain, and then scanned on GCOS 3000 (Affymetrix).

The data are analyzed using Software Genespring V7.3. A t test on normalized intensity followed by ratio change (ratio of normalized intensity ≧2 or ≦−2) is used to generate the gene list with significant change in gene expression profile. In this study, MSCs from 2 patients in duplicate are used.

### Example 7

### Transplantation of Human MSCs Into Rats and Histological Evaluation

Male Sprague Dawley (SD) rats (500 g) are anesthetized using an intraperitoneal injection of a mixture ketamine (10 mg/100 g) and xylazine (1 mg/100 g).

An anterior midline incision is made through the skin of the knee. The knee joints are opened via the parapatellar-medial approach and the patella is everted. An osteochondral defect (1.5 mm in diameter and 1.5 mm in depth) is made in the patellar groove of the distal femur. Three rats received pellets with ZNF145 overexpressing hMSCs transplanted into right knee and control hMSCs into left knees. The pellets from 3×1̂5 ZNF145-overexpressing hMSCs pelleted are induced into chondrocyte differentiation in vitro for 1 week before transplantation (Johnstone et al., 1998).

Defects with pellets from no insert control MSCs are used as control. The recipient animals received daily cubcutaneous injections of Cyclosporine (14 mg/kg, Novartis Pharma A G, Basel, Switherland) immediately after surgery.

At 6 weeks after surgery, three rats from each group are sacrificed each time. The distal femurs with defects are collected, fixed in 10% buffered formation, and the tissues decalcified and cut into 5 μm section. Staining is performed with hematoxyliln/eosin, alcian blue for cartilage proteoglycans and Co12A1 immunostain.

Each sample is graded according to the histological scale described by Wakitani et al (1994). The scale consisted of five categories: cell morphology, matrix staining, surface regularity, thickness of cartilage, and integration of donor with host cartilage. The scores ranged from 0 (normal articular cartilage) to 14 (no cartilaginous tissue).

### Example 8

### Results: Expression Pattern of ZNF145 During In Vitro Chondrogenesis

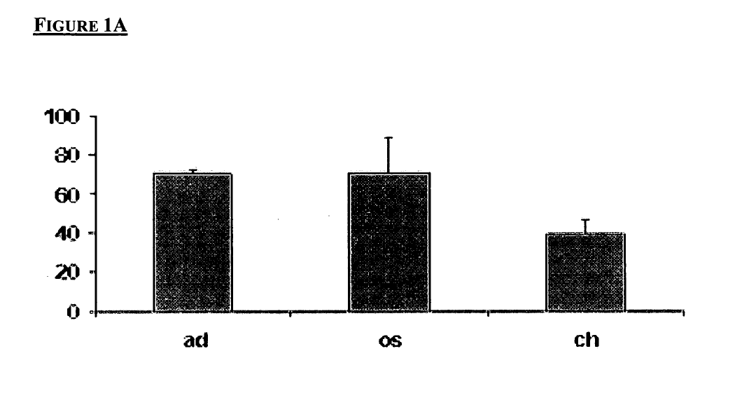

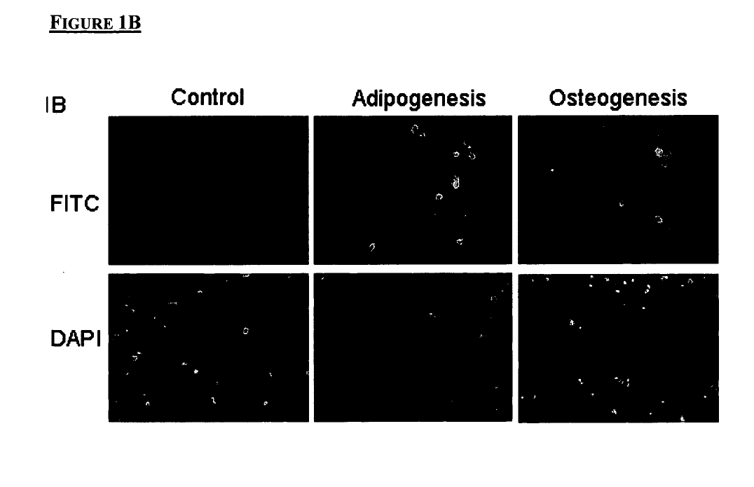

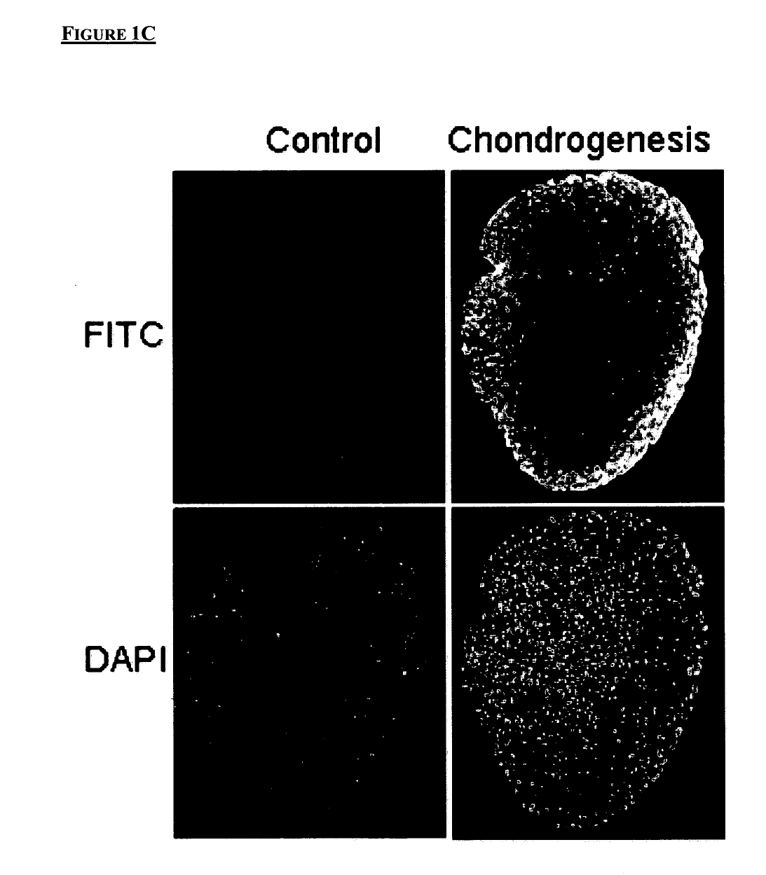

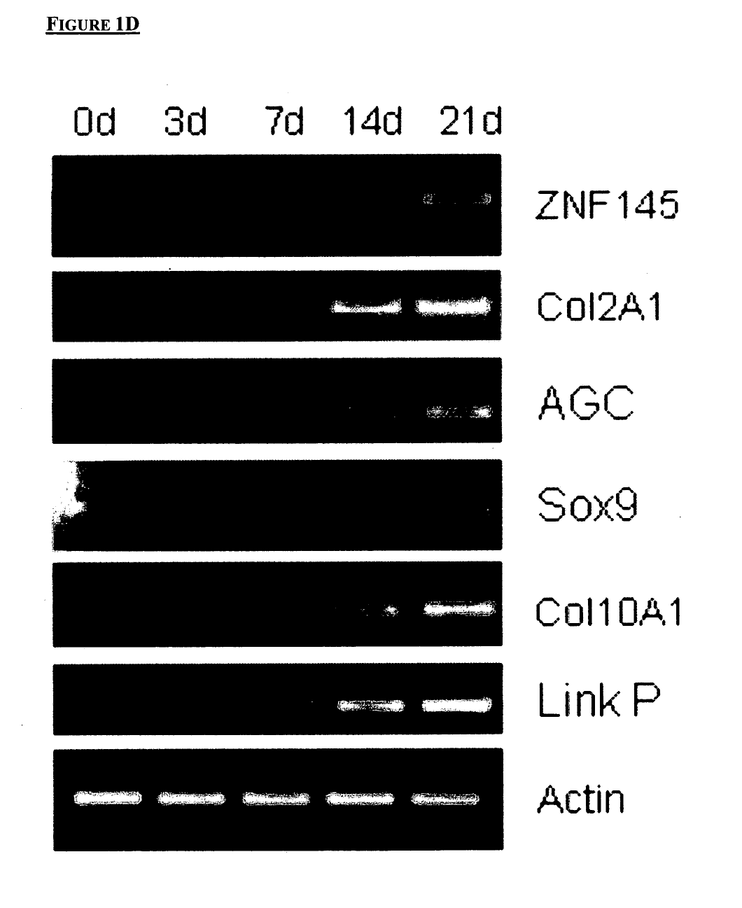

Quantitative data by real time PCR (FIG. 1A) shows that ZNF145 is commonly upregulated during three lineages differentiation of MSCs at early and late stages.

Immunofluoresence against ZNF145 shows that ZNF145 is upregulated during three lineages of differentiation and localized in nuclei whereas ZNF145 is not expressed in undifferentiated MSCs (FIG. 1B and FIG. 1C).

### Example 9

### Results: Effect of ZNF145 Knockdown on Three Lineages of Differentiation

ZNF145 is shown as common upregulated genes during 3 lineages of differentiation of MSCs.

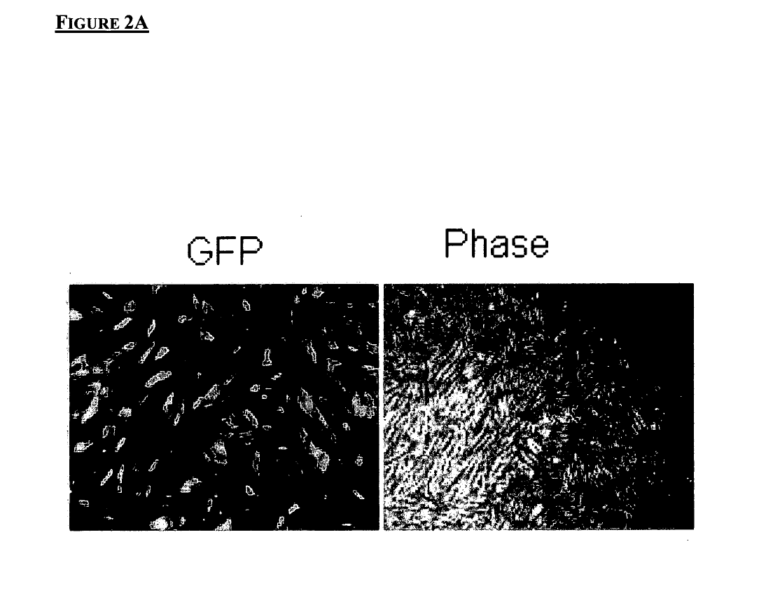

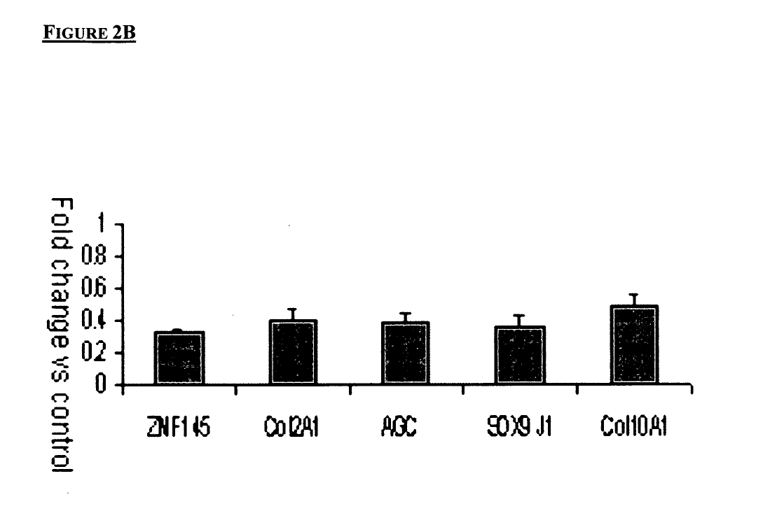

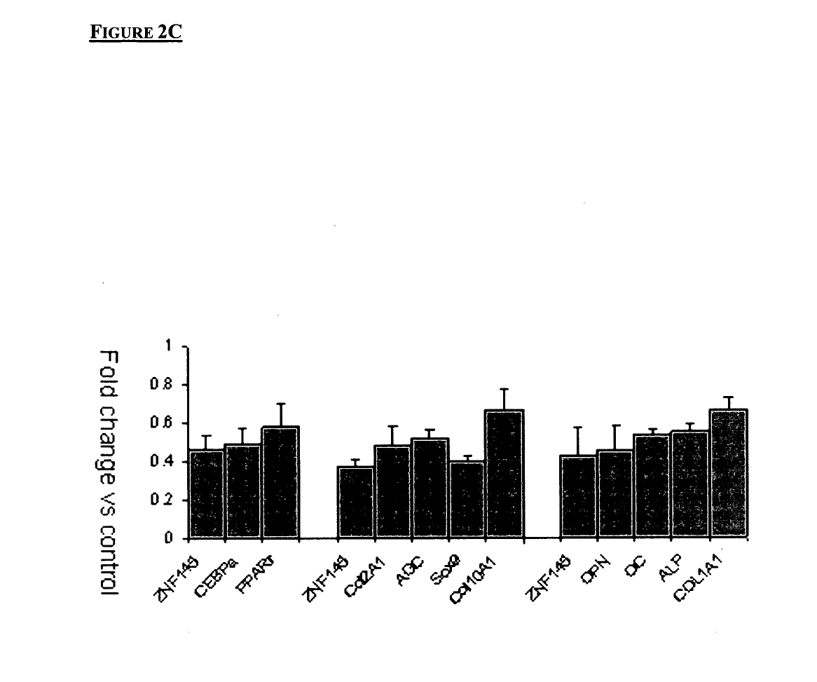

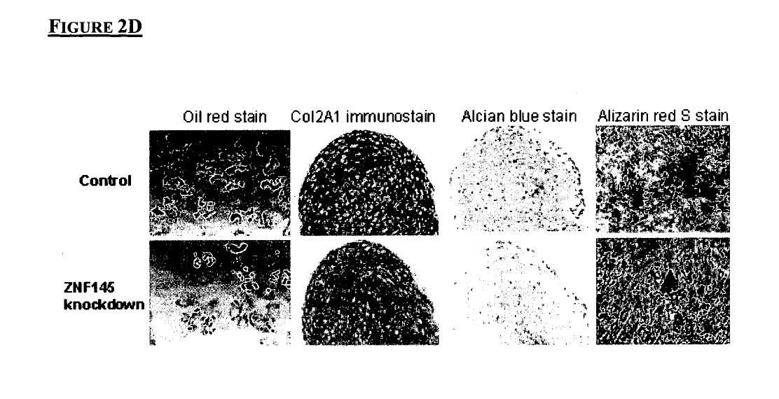

Two shRNA targeting ZNF145 are constructed shown under Experimental Procedure. ShRNA targeting ZNF145 is efficiently introduced into MSCs by lentiviral pLL3.7 (FIG. 2A). Gene silencing of ZNF145 by siRNA downregulated three lineages of markers quantified by real time PCR (FIG. 2B and FIG. 2C). The results show that ZNF145 plays an important role in differentiation of MSCs. These are consistent with stain for three lineages of differentiation.

ZNF145 knockdown MSCs shows decreased oil red stain for oil droplet in adipogenesis, Col2a1 for major collagen and alcian blue for sulfated proteoglycan matrix in chondrogenesis, and alizarin red stain for calcium deposit in osteogenesis (FIG. 2D).

### Example 10

### Results: Effect of ZNF145 Overexpression on Chondrogenesis and Osteogenesis

To assess to effects of ZNF145 overexpression on differentiation of MSCs, MSCs are infected with lentivirus for stable ZNF145 overexpression

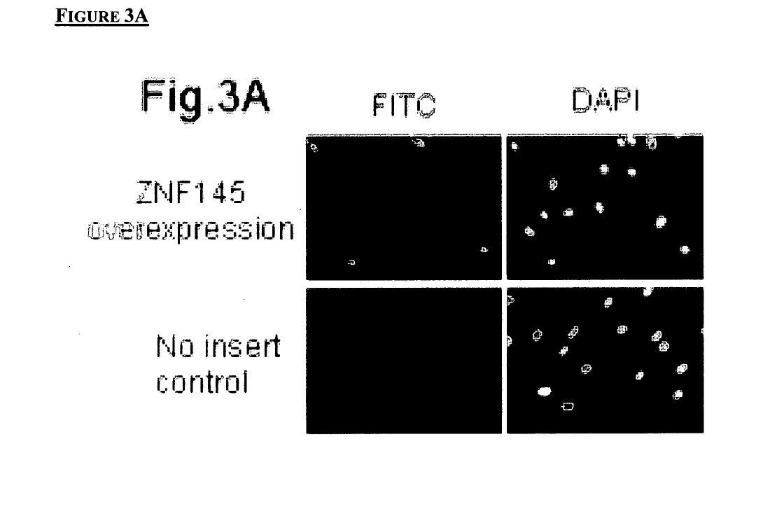

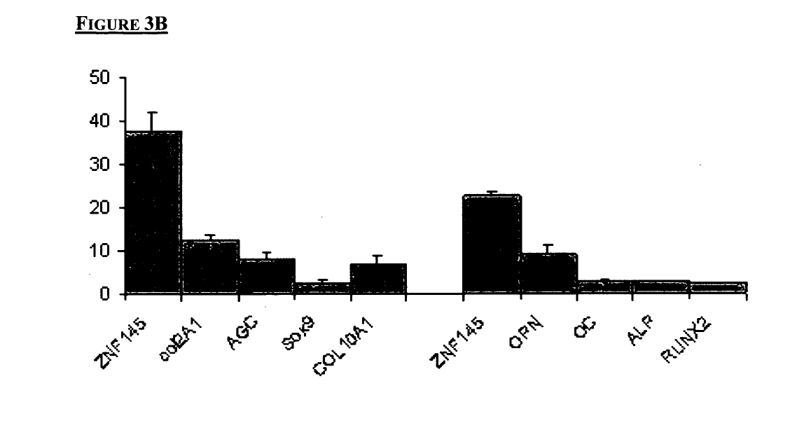

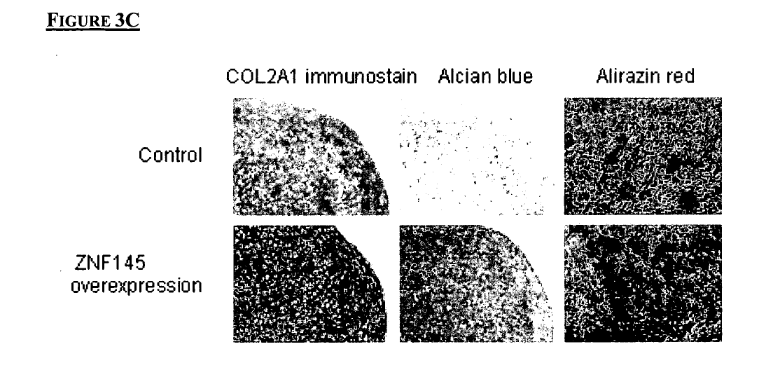

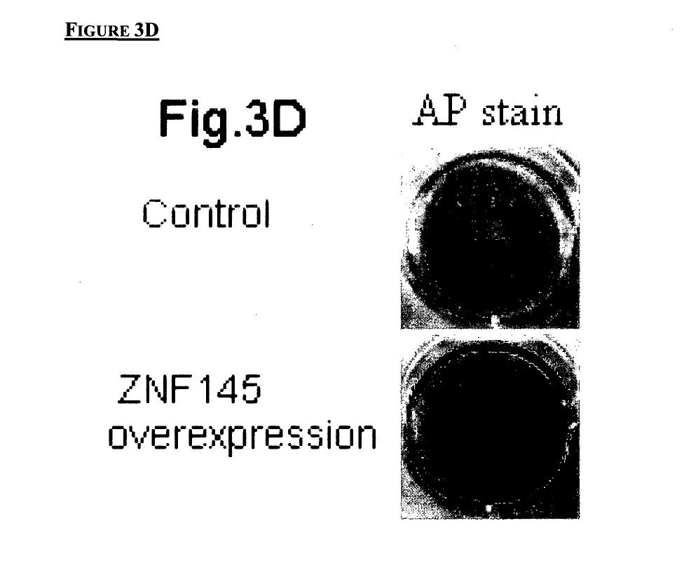

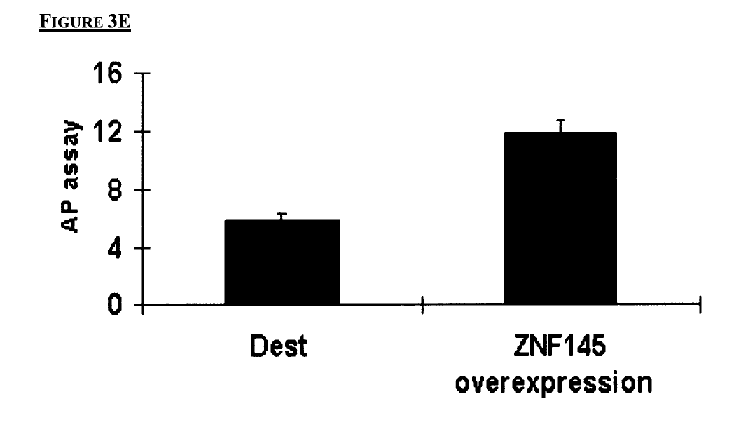

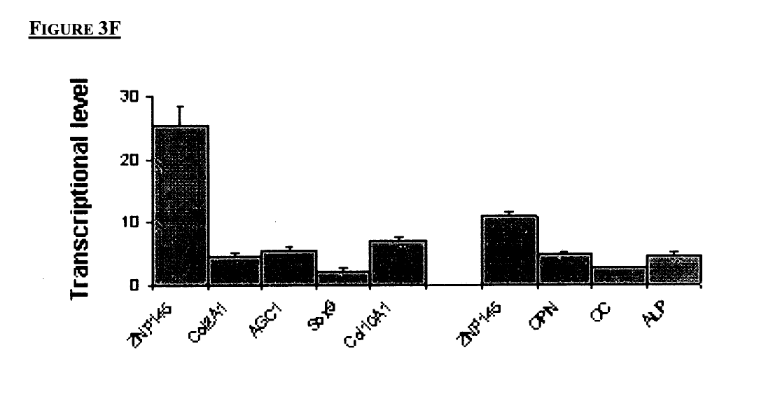

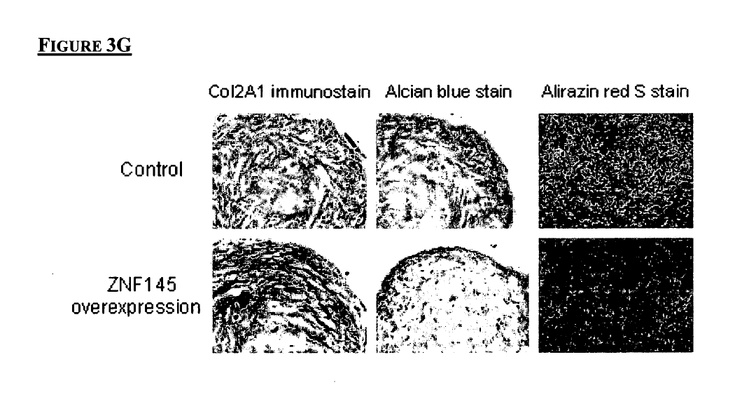

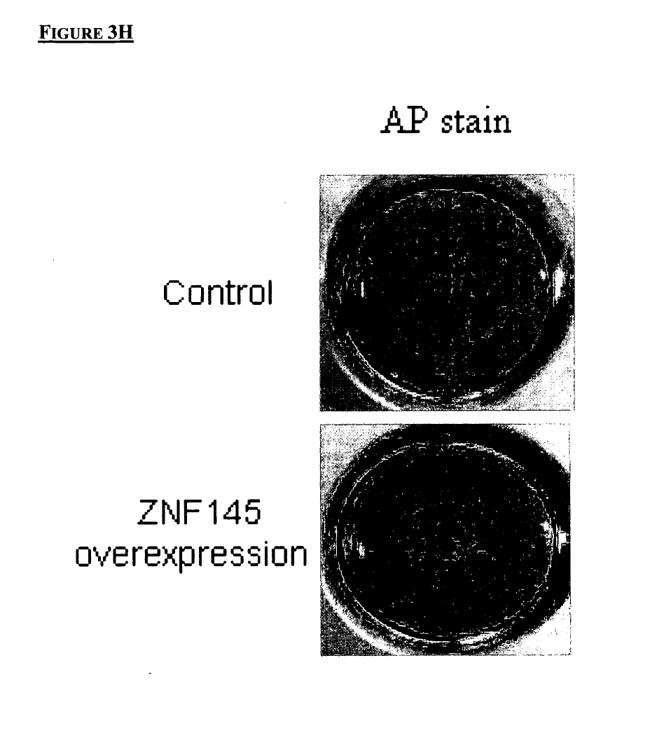

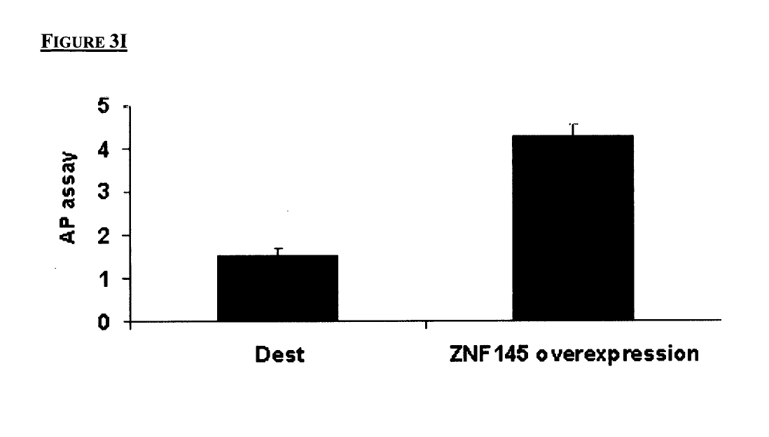

Immunostaining shows that ZNF145 is overexpressed in nuclei of MSCs whereas ZNF145 is not expressed in undifferentiated MSCs (FIG. 3A).

Then ZNF145-overexpressing MSCs are induced into chondrogenesis for 28 days under pellet culture and osteogenesis for 14 days.

Overexpression of ZNF145 in MSCs promotes the expression of col2A1 by 12.64-fold, aggrecan by 7.92-fold, col10A1 by 7.5-fold and sox9 by 2.45-fold in chondrogenesis (FIG. 3B), showing ZNF145 promotes chondrogenesis.

This finding is further verified by immunostain for col2A1 stain and alcian blue for proteoglycan in cartilage (FIG. 3C).

In osteogenesis, upregulation of osteocalcin, alkaline phosphatase and col1A1 is observed in ZNF145-overexpressing MSCs compared with no insert control (FIG. 3B). This is consistent with alizarin red for calcium deposition (FIG. 3C) and AP stain for alkaline phosphatase (FIG. 3D and FIG. 3E) in osteogenesis, showing ZNF145 overexpression improves osteogenesis.

### Example 10A

### ZNF145 Over-Expression Improves Chondrogenesis and Osteogenesis of MSC Cell Line

Primary MSCs pose a problem with limited life span and variance from donor to donor. To overcome these disadvantage with primary MSCs, MSC cell line is generated with combination of hTERT and antigen large T from SV40 via retroviral system. MSC cell line displayed similar surface antigen profile to primary MSCs and had differentiation potential towards three lineages (adipocytes, chondrocytes and osteocytes).

To test tumorigenesis of MSC cell line, 5×10⁶ MSC cell line is subcutaneously transplanted into 5 nude mice and 5 NOG mice per site, no tumors are observed at week 12.

Our findings showed ZNF145 overexpression had similar effects in MSC cell line to primary MSCs, ZNF145overexpression in MSC cell line enhanced the expression of chondrogenic and osteogenic markers (FIG. 3F), consistent with enhanced Col2A1 by immunostaining against Col2A1 and Alcian blue stain for sulfated proteoglycan matrix in cartilage differentiation and enhanced calcium deposits by Alizarin red stain (FIG. 3G) and alkaline phosphatase by AP stain (FIG. 3H) and AP assay (FIG. 3I) in osteogenesis.

### Example 11

### Results: Targets of ZNF145 in MSCs

To elucidate the mechanism underlying effects of ZNF145 overexpression on MSCs, we overexpress ZNF145 in two patient-derived MSCs and then check its targets in MSCs in duplicate by microarray.

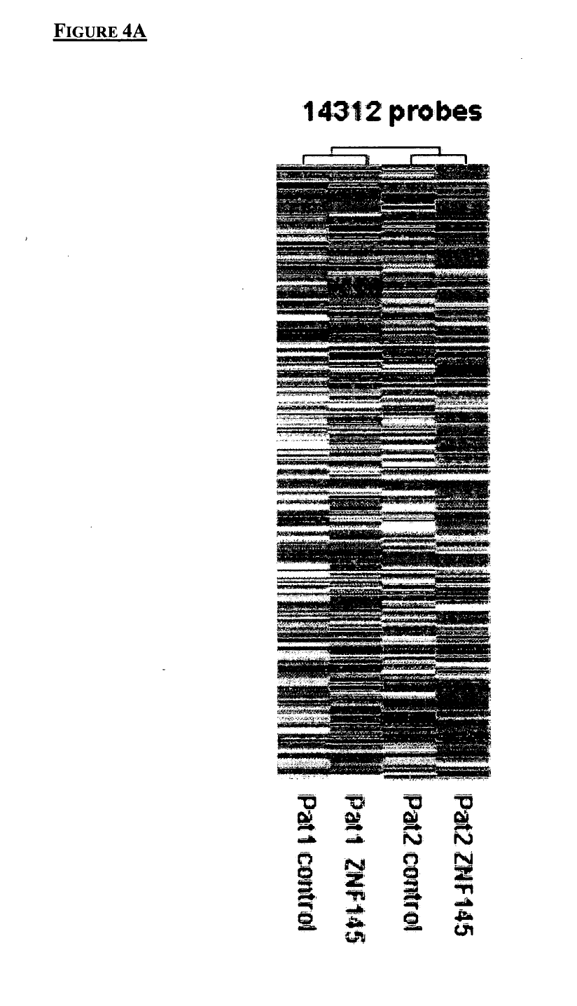

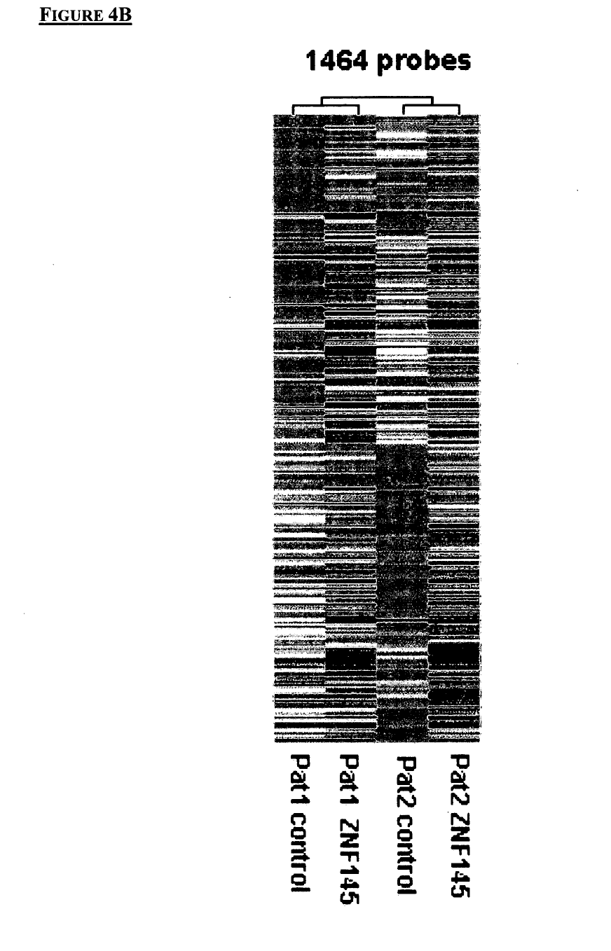

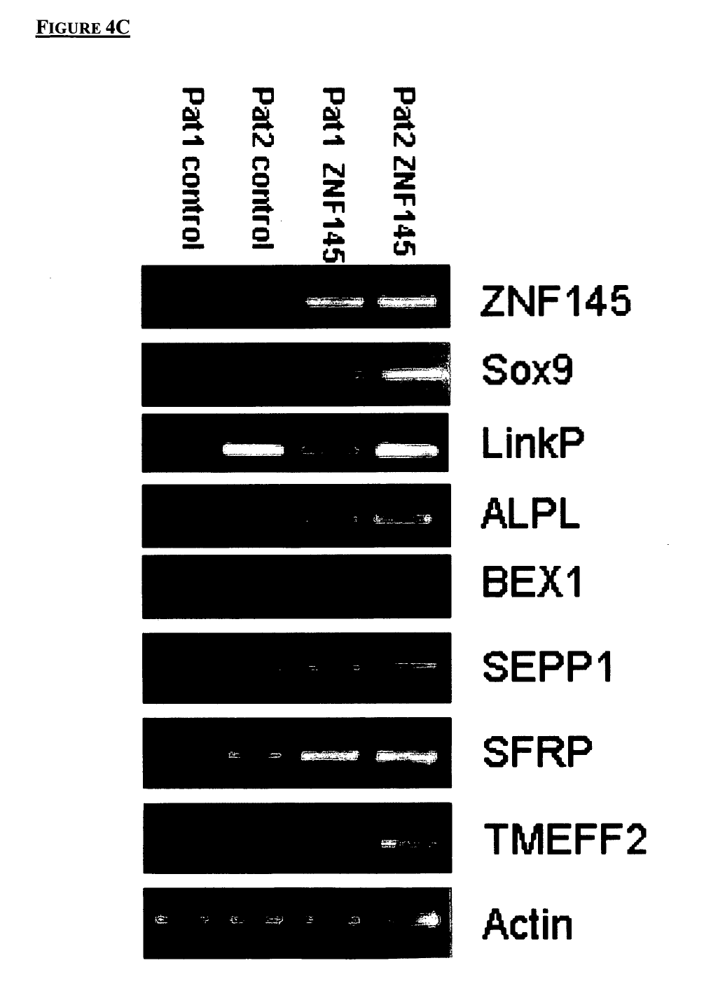

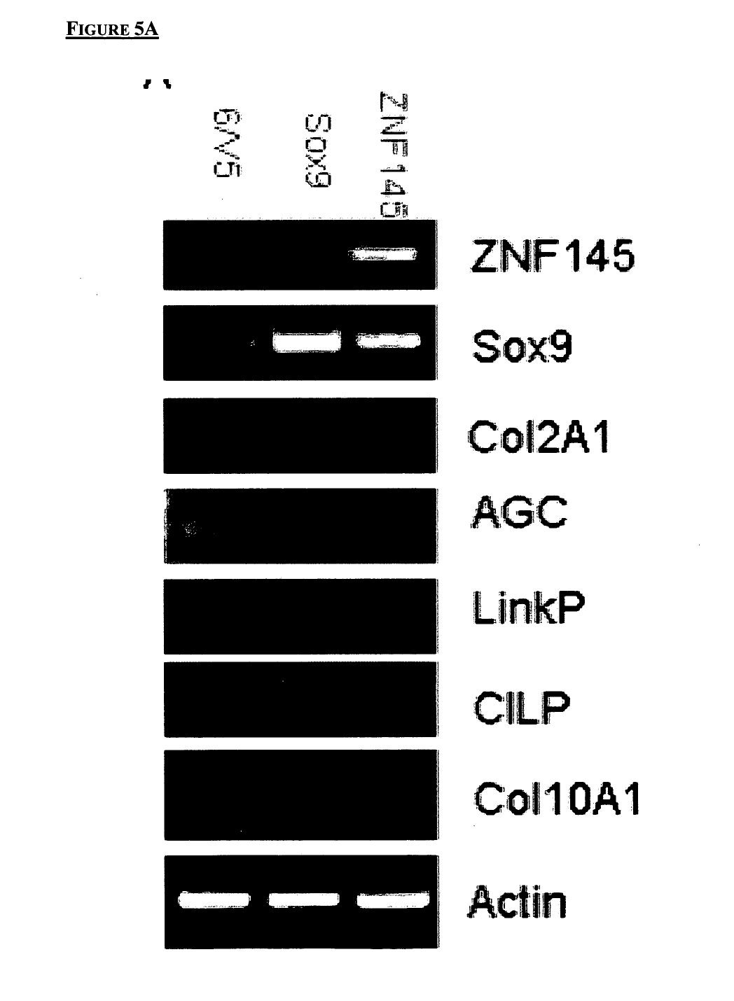

Our data shows that 423 genes are upregulated by ZNF145 overexpresison whereas 678 genes are downregulated by ZNF145 overexpresion in undifferentiated MSCs (FIG. 4A).

Two patient-derived MSCs show similar expression pattern upon ZNF145 overexpression (FIG. 4B).

The expression patterns of selected genes from parallel samples analyzed by microarrays are subsequently compared by RT-PCR for validation. RT-PCR assays are consistent with the microarray data (FIG. 4C).

### Example 12

### Results: ZNF145 Regulated Chondrogenesis as an Upstream Regulator of Sox9

Sox9 is master regulator during chondrogenesis. To understand how ZNF145 functions in chondrogenesis, it is crucial to determine the relationship between ZNF145 and Sox9.

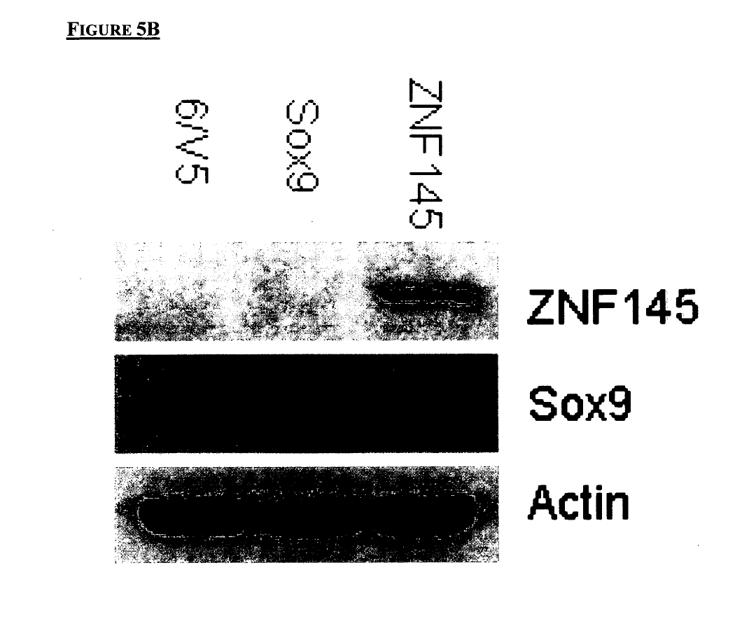

Sox9 and ZNF145 are introduced into MSCs. Overexpression of ZNF145 in MSCs enhances the expression of Sox9 whereas overexpression of Sox9 does not enhance the expression of ZNF145 in RNA (FIG. 5A) and protein level (FIG. 5B). This finding suggests that ZNF145 is an upstream regulator of Sox9.

### Example 13

### ZNF145 Improved Repair of Cartilage Defect in an In Vivo Rat Model

To assess whether ZNF145-overexpressing MSCs would improve the quality of the repair of a cartilage defect in vivo, we compare MSCs overexpressing ZNF145 with no insert control MSCs in which had been subjected to in vitro chondrogenesis for 7 days under pellet culture and then transplanted into osteochondral defects of rat knees.

Immediately after surgery, the recipient animals received daily subcutaneous injections of cyclosporine (14 mg/kg). Six weeks after transplantation, the defects are filled with reparative tissue that resembled hyaline cartilage.

The superficial layers from ZNF145-MSC transplants had more intense matrix staining compared with control MSCs. At higher magnification, the cells resembled well differentiated chondrocytes and are surrounded by metachromatic matrix.

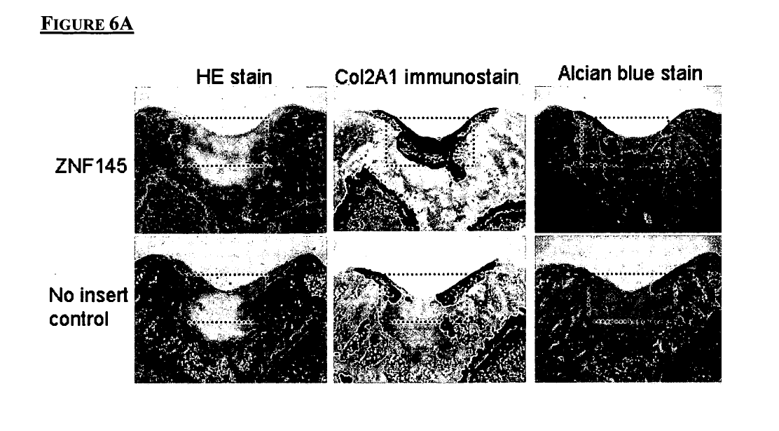

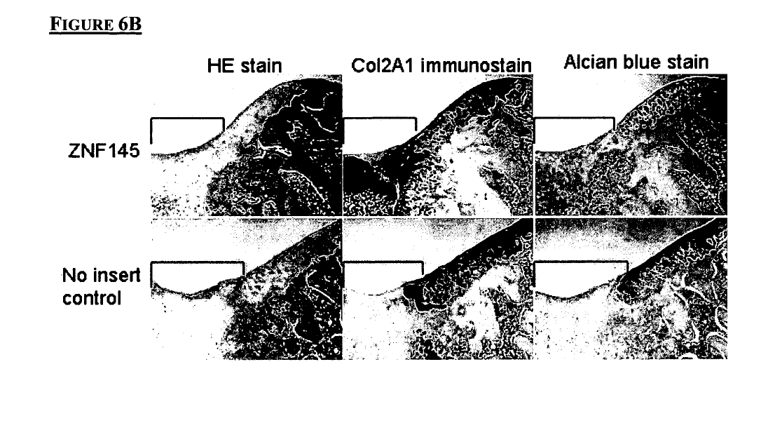

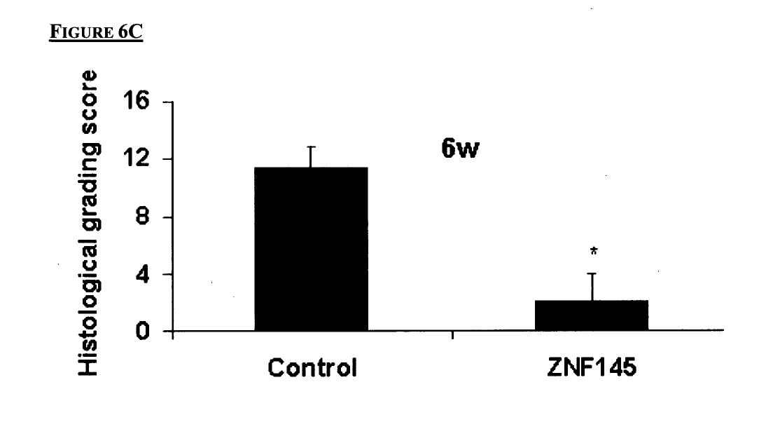

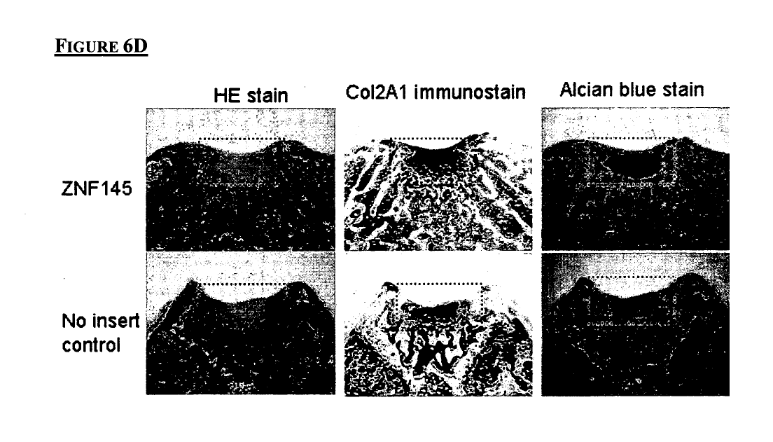

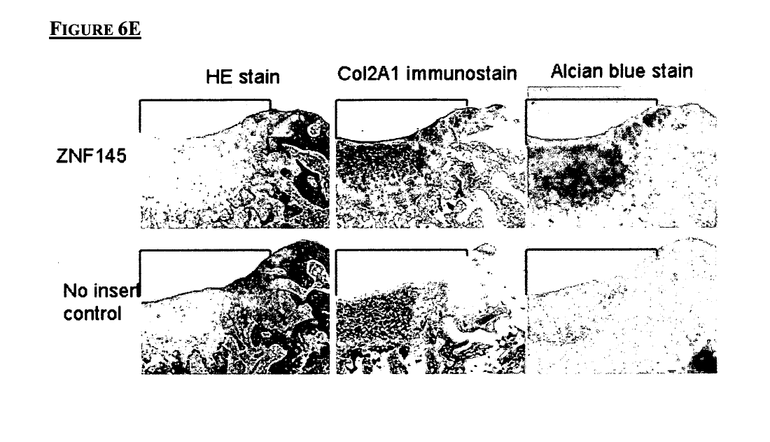

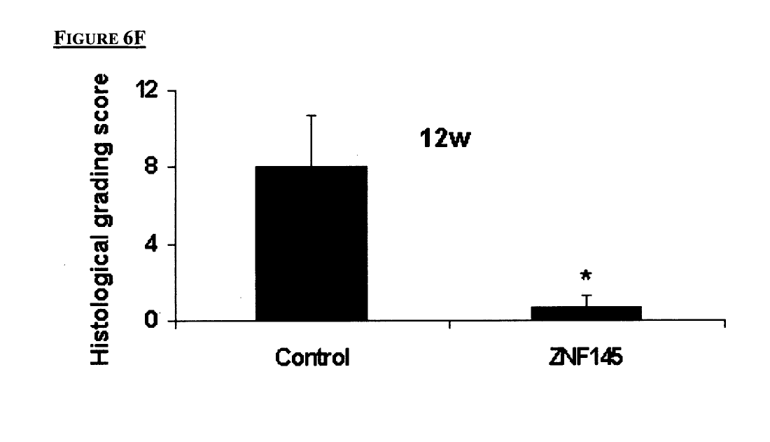

The ZNF145 group showed continuous and similar Alcian blue stain for sulfated proteoglycan matrix and Col2A1 immunostaining for major collagen of cartilage to adjacent cartilage whereas the no insert control MSCs group showed discontinuous Alcian blue staining and Col2A1 immunostaining at defect sites. Mostly importantly, cartilage from ZNF145-MSCs integrated well to both edges of adjacent cartilage (FIGS. 6A and 6B).

Histological grading scores are determined to compare the repair tissues between the ZNF145 and the no insert control groups according to Wakitani et al (1994) based on cell morphology, matrix-staining, surface regularity, thickness of cartilage and integration of donor with host adjacent cartilage.

The grading scores of ZNF145 group are significantly better than those of no insert control group (FIG. 6C). These results showed that ZNF145-overexpresing MSCs repaired cartilage defects much better and earlier than control group, the ZNF145 group possessed superior cartilage.

Similar to reparative effects at 6 weeks, ZNF145 group at 12 weeks show better reparative effects compared with control MSCs (FIGS. 6D and 6E). The grading scores of the ZNF145 group at 12 weeks are much better than the control group (FIG. 6F)

These results show ZNF145 improved the quality of repair of cartilage defects and is able to do it better and earlier. These findings suggest that ZNF145 therapy may be a useful strategy for cartilage regeneration and repair.

### Example 15

### Generation of Mesenchymal Stem Cell Lines

A viral protein may also be expressed in addition to the telomerase in order to immortalise the mesenchymal stem cell lines. The above experiments are repeated to produce mesenchymal stem cell lines which expresse (a) hTERT and SV-40 Large T antigen; (b) hTERT+HPV E7; (c) hTERT+HPV E6+HPV E7; or (d) hTERT+bmi-1+HPV E6.

A brief procedure of generation of mesenchymal stem cell lines comprises the steps of: (a) generating lentiviral expression vectors, as described in detail above; (b) generating virus separately (see Examples—Materials and Methods); (c) coinfecting bone marrow-MSCs (or other types of MSCs including adipose tissue-MSC, ES-MSCs, etc) with virus or infecting MSCs one after another according to the above combinations; (d) continuing passage to test if generated MSCs with the above combinations are immortal lines and also check their differentiation potential including cartilage differentiation.

Results

A mesenchymal stem cell line expressing hTERT+Large T antigen is produced as described above. Data shows that this combination can prolong life span of bone marrow-MSC to 25th passage where no gene control MSCs undergo senescence at passage 15. The generated MSCs show good morphology of MSCs and 3 lineages of differentiation towards bone, cartilage and fat differentiation.

### REFERENCES

References 1 to 13 relate to the Background:

1. Buckwalter J A. Articular cartilage injuries. Clin Orthop, 2002:21-37.

2. Martin J A, Buckwalter J A. Aging, articular cartilage chondrocyte senescence and osteoarthritis. Biogerontology 2002; 3:257-264.

3. Weissman I L. Translating stem and progenitor cell biology to the clinic: barriers and opportunities. Science 2000; 287:1442-1446.

4. Pittenger M F, Mackay A M, Beck S C, et al. Multilineage potential of adult human mesenchymal stem cells. Science 1999; 284:143-147.

5. Zuk P A, Zhu M, Mizuno H, et al. Multilineage cells from human adipose tissue: implications for cell-based therapies. Tissue Eng 2001; 7:211-228.

6. Pountos I, Jones E, Tzioupis C, et al. Growing bone and cartilage: The role of mesenchymal stem cells. J Bone Joint Surg Br 2006; 88:421-426.

7. Horwitz E M, Gordon P L, Koo W K, et al. Isolated allogeneic bone marrow-derived mesenchymal cells engraft and stimulate growth in children with osteogenesis imperfecta: Implications for cell therapy of bone. Proc Natl Acad Sci USA 2002; 99:8932-8937.

8. Chamberlain J R, Schwarze U, Wang P R, et al. Gene targeting in stem cells from individuals with osteogenesis imperfecta. Science 2004; 303: 1198-1201.

9. Arinzeh T L, Peter S J, Archambault M P, et al. Allogeneic mesenchymal stem cells regenerate bone in a critical-sized canine segmental defect. J Bone Joint Surg Am 2003; 85-A: 1927-1935.

10. Quarto R, Thomas D, Liang C T. Bone progenitor cell deficits and the age-associated decline in bone repair capacity. Calcif Tissue Int 1995; 56:123-129.

11. D'Ippolito G, Schiller P C, Ricordi C et al. Age-related osteogenic potential of mesenchymal stromal stem cells from human vertebral bone marrow. J Bone Miner Res 1999; 14:1115-1122.

12. Muraglia A, Cancedda R, Quarto R. Clonal mesenchymal progenitors from human bone marrow differentiate in vitro according to a hierarchical model. J Cell Sci 2000; 113:1161-1166.

13. Guilak F, Lott K E, Awad H A, et al. Clonal analysis of the differentiation of potential of human adipose-derived adult stem cells. J Cell Physiol 2006; 206:229-237.

The following references refer to the description (not including the Background)

Akiyama H, Chaboissier M C, Martin J F, Schedl A, De Crombrugghe B. The transcription factor Sox9 has essential roles in successive steps of the chondrocyte differentiation pathway and is required for expression of Sox5 and Sox6. Genes Dev. 2002, 16:2813-2828.

Barna M, Hawe N, Niswander L, Pandolfi P P. Plzf regulates limb and axial skeletal patterning. Nature, 2000, 25:166-172.

Bell D M, Leung K K, Wheatley S, Ng L J, Zhou S, Ling K W, Sham M H, Koopman P, Tam P P, Cheah K S. Sox9 directly regulates the type-II collagen gene. Nature, 1997, 16:174-178.

Bi W, Deng J M, Zhang Z, Behringer R R, de Crombrugghe B. Sox9 is required for cartilage formation Nat Genet., 1999, 22:85-89.

Bridgewater L C, Lefebvre V, De Crombrugghe B. Chondrocyte-specific enhancer elements in the Col11a2 gene resemble the Col2a1 tissue-specific enhancer. J Biol Chem, 1998, 273:14998-15006.

Buckwalter J A. Articular cartilage injuries. Clin Orthop, 2002:21-37.

Chen Z, Brand N J, Chen A, Chen S J, Tong J H, Wang Z Y, Waxman S, Zelent A. Fusion between a novel Krüppel-like zinc finger gene and the retinoic acid receptor-alpha locus due to a variant t(11;17) translocation associated with acute promyelocytic leukaemia. EMBO J, 1993, 12(3):1161-1167.

Cook M, Gould A, Brand N, Davies J, Strutt P, Shaknovich R, Licht J, Waxman S, Chen Z, Gluecksohn-Waelsch S, Krumlauf R, Zelent A. Expression of the zinc-finger gene PLZF at rhombomere boundaries in the vertebrate hindbrain. Proc Natl Acad Sci USA, 1995, 92:2249-2253.

Costoya J A, Hobbs R M, Barna M, Cattorettti G, Manova K, Sukhwani M, Orwig K E, Wolgemuth D J, Pandolfi P P. Essential role of Plzf in maintenance of spermatogonial stem cells. Nature Genet, 2004, 36: 653-659.

Eaves C J, Cashman J D, Kay R J, Dougherty G J, Otsuka T, Gaboury L A, Hogge D E, Lansdorp P M, Eaves A C, Humphries R K. Mechanisms that regulate the cell cycle status of very primitive hematopoietic cells in long-term human marrow cultures. II. Analysis of positive and negative regulators produced by stromal cells within the adherent layer. Blood, 1991, 78:110-117.

Foster J W, Dominguez-Steglich M A, Guioli S, Kwok C, Weller P A, Stevanovic M, Weissenbach J, Mansour S, Young I D, Goodfellow P N, Brook J D, Schafer A J. Campomelic dysplasia and autosomal sex reversal caused by mutations in an SRY-related gene. Nature, 1994, 372: 525-530.

Furumatsu T, Tsuda M, Taniguchi N, Tajima Y, Asahara H. Smad3 induces chondrogenesis through the activation of SOX9 via CREB-binding protein/p300 recruitment. J Biol Chem, 2005, 280:8343-8350.

Huang W, Chung U, Kronenberg H M, de Crombrugghe B. The chondrogenic transcription factor Sox9 is a target of signaling by the parathyroid hormone-related peptide in the growth plate of endochondral bones. Proc Natl Acad Sci, 2001, 98:160-165.

Ikeda R, Yoshida K, Tsukahara S, et al. The promyelotic leukemia zinc finger promotes osteoblastic differentiation of human mesenchymal stem cells as an upstream regulator of CBFA1. J Biol Chem 2005; 280:8523-8530.

Ikegawa S and Kou I. SOX9-dependent and -independent transcriptional regulation of human cartilage link protein. J Biol Chem, 2004, 279: 50942-50948.

Johnstone B, Hering T M, Caplan A I, Goldberg V M, Yoo J U. In vitro chondrogenesis of bone marrow-derived mesenchymal progenitor cells. Exp Cell Res 1998; 238(1): 265-272.

Kawakami Y, Tsuda M, Takahashi S, Taniguchi N, Esteban C R, Zemmyo M, Furumatsu T, Lotz M, Belmonte J C I, Asahara H. Transcriptional coactivator PGC-1α regulates chondrogenesis via association with Sox9. Proc Natl Acad Sci, 2005, 102:2414-2419.

Lefebvre V, Huang W, Harley V R, Goodfellow P N, De Crombrugghe B. SOX9 is a potent activator of the chondrocyte-specific enhancer of the pro alpha1 (II) collagen gene. Mol Cell Biol, 1997, 17: 2336-2346.

Liu T M, Martina M, Hutmacher D W, Hui J H P, Lee E H, Lim B. Identification of common pathways mediating differentiation of bone marrow and adipose tissues derived human mesenchymal stem cells (MSCs) into three mesenchymal lineages. Stem cells, 2007, 25:250-260.

Martin J A, Buckwalter J A. Aging, articular cartilage chondrocyte senescence and osteoarthritis. Biogerontology 2002; 3:257-264.

Melnick A M, Westendorf J J, Polinger A, Carlile G W, Arai S, Ball H J, Lutterbach B, Hiebert S W, Licht J D. The ETO protein disrupted in t(8;21)-associated acute myeloid leukemia is a corepressor for the promyelocytic leukemia zinc finger protein. Mol Cell Biol., 2000, 20(6): 2075-2086.

Murakami S, Kan M, McKeehan W L, de Crombrugghe B. Up-regulation of the chondrogenic Sox9 gene by fibroblast growth factor is mediated by the mitogen-activated protein kinase pathway. Proc Natl Acad Sci, 2000, 97:1113-1118.

Ng L J, Wheatley S, Muscat G E, Conway-Campbell J, Bowles J, Wright E, Bell D M, Tam P P, Cheah K S, Koopman P. SOX9 binds DNA, activates transcription, and coexpresses with type II collagen during chondrogenesis in the mouse. Dev Biol, 1997, 183:108-121.

Ng L J, Wheatley S, Muscat G E, Conway-Cambell J, Bowles J, Wright E, Bell D M, Tam P P, Cheah K S, Koopman P. SOX9 binds DNA, activates transcription, and coexpresses with type II collagen during chondrogenesis in the mouse. Dev Biol. 1997, 183(1):108-121.

Sekiya Γ, Tsuji K, Koopman P, Watanabe H, Yamada Y, Shinomiya K, Nifuji A, Noda M. SOX9 enhances aggrecan gene promoter/enhancer activity and is up-regulated by retinoic acid in a cartilage-derived cell line, TC6. J Biol Chem, 2000, 275: 10738-10744.

Sekiya I, Vuoristo J T, Larson B L, Prockop D J. In vitro cartilage formation by human adult stem cells from bone marrow stroma defines the sequence of cellular and molecular events during chondrogenesis. Proc Natl Acad Sci 2002; 99:4397-4402.

Shaknovich R, Yeyati P L, Ivins S, Melnick A, Lempert C, Waxman S, Zelent A, Licht J D. The promyelocytic leukemia zinc finger protein affects myeloid cell growth, differentiation, and apoptosis. Mol Cell Biol, 1998, 18:5533-5545.

Wakitani S, Goto T, Pineda S J, Young R G, Mansour J M, Caplan A I, Goldberg V M. Mesenchymal cell-based repair of large, full-thickness defects of articular cartilage. J Bone Joint Surg Am, 1994, 76(4):579-592.

Wagner T, Wirth J, Meyer J, Zabel B, Held M, Zimmer J, Pasantes J, Bricarelli F D, Keutel J, Hustert E, Wolf U, Tommerup N, Schempp W, Scherer G. Autosomal sex reversal and campomelic dysplasis are caused by mutations in and around the SRY-related gene SOX9. Cell, 1994, 79: 1111-1120.

Xie W F, Zhang X, Sakano S. Lefebvre V, Sandell L J. Trans-activation of the mouse cartilage-derived retinoic acid-sensitive protein gene by Sox9. J Bone Miner Res, 1999, 14: 757-763.

Zhao Q, Eberspaecher H, Lefebvre V, de Crombrugghe B. Parallel expression of Sox9 and Col2a1 in cells undergoing chondrogenesis. Dev Dyn, 1997, 209:377-386.

Zuk P A, Zhu M, Ashjian P, De Ugarte D A, Huang J I, Mizuno H, Alfonso Z C, Fraser J K, Benhaim P, Hedrick M H. Human adipose tissue is a source of multipotent stem cells. Mol Biol Cell 2002; 13:4279-4295.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1-16. (canceled)

17. A chondrogenic progenitor cell which has been engineered, or for which an ancestor thereof has been engineered, by causing entry into the cell of a ZNF145 expression vector that encodes a polynucleotide comprising chondrogenic activity, in which the engineered chondrogenic progenitor cell displays increased expression or activity of a ZNF145 polypeptide as compared to a cell that has not been so engineered.

18. The cell of claim 17 wherein chondrogenesis is increased.

19. The cell of claim 17 wherein said cell is a mesenchymal stem cell.

20. The cell of claim 17 which exhibits:
a. Enhanced expression of a chondrogenic marker;
b. Enhanced secretion of cartilage proteoglycans;
c. Improved ability to repair a cartilage, bone, or ligament defect; or
any combination of a, b, and c, as compared to a chondrogenic progenitor cell which has not been so engineered.

21. The cell of claim 20 wherein said chondrogenic marker is selected from the group consisting of collagen type 2 (COL2A1), aggrecan, col10A1, and Sox9.

22. The cell of claim 17 wherein said cell has been induced to chondrocyte differentiation.

23. The cell of claim 17 wherein said cell has been further engineered to increase expression or activity of one or more genes selected from the group consisting of Nanog, Oct4, telomerase, SV40 large T antigen, HPV E6, HPV E7 or Bmi-1.

24. A cell line comprising a cell according to claim 17.

25. The cell line of claim 24 wherein said cell line is immortalized.

26. A pharmaceutical composition comprising a pharmaceutically acceptable carrier and a pro-chondrogenic factor selected from the group consisting of: a chondrogenic progenitor cell which has been engineered, or for which an ancestor thereof has been engineered, by causing entry into the cell of a ZNF145 expression vector that encodes a polynucleotide comprising chondrogenic activity, to increase expression or activity of ZNF145; a cell line wherein the cell has been engineered, or wherein an ancestor thereof has been engineered, to increase expression or activity of ZNF145; a nucleic acid comprising a ZNF145 sequence that encodes a polypeptide having chondrogenic activity; and a polypeptide comprising a ZNF145 sequence having chondrogenic activity.

27. A method of promoting cartilage, bone, or ligament repair or inducing repair or regeneration of chondral tissue, the method comprising enhancing the expression or activity of ZNF145 in a chondrogenic progenitor cell.

28. The method of claim 27 wherein said enhancing expression or activity of ZNF145 is accomplished using a pro-chondrogenic factor selected from the group consisting of: a chondrogenic progenitor cell which has been engineered, or for which an ancestor thereof has been engineered, by causing entry into the cell of a ZNF145 expression vector that encodes a polynucleotide comprising chondrogenic activity, to increase expression or activity of ZNF145; a cell line wherein the cell has been engineered, or wherein an ancestor thereof has been engineered, to increase expression or activity of ZNF145; a nucleic acid comprising a ZNF145 sequence that encodes a polypeptide having chondrogenic activity; and a polypeptide comprising a ZNF145 sequence having chondrogenic activity.

29. A method of treating a condition or disease comprising contacting a tissue or administering to an individual a pro-chondrogenic factor selected from the group consisting of: a chondrogenic progenitor cell which has been engineered, or for which an ancestor thereof has been engineered, by causing entry into the cell of a ZNF145 expression vector that encodes a polynucleotide comprising chondrogenic activity, to increase expression or activity of ZNF145; a cell line wherein the cell has been engineered, or wherein an ancestor thereof has been engineered, to increase expression or activity of ZNF145; a nucleic acid comprising a ZNF145 sequence that encodes a polypeptide having chondrogenic activity; and a polypeptide comprising a ZNF145 sequence having chondrogenic activity.

30. The method of claim 29 wherein said treating a condition or disease is selected from the group consisting of repair or regeneration of chondral tissue or repair of a bone or ligament defect.

31. The method of claim 29 wherein said disease or condition is selected from the group consisting of a disease, damage, disorder, or injury associated with cartilage, a bone or ligament defect, a traumatic injury, an age-related degenerative disease or a degenerative joint disease.

32. The method of claim 29 wherein said chondrogenic progenitor cell engineered to increase expression or activity of ZNF145 comprises an expression construct that increases the expression or activity of ZNF145.

33. A method of treating a disease in an individual comprising up-regulating the expression or activity of ZNF145 in chondrogenic progenitor cells in an individual in need of such treatment.

34. The method of claim 33 wherein said chondrogenic progenitor cells are cells with increased ZNF145 expression or activity that are administered to the individual.
